(19) United States
(12) Patent Application Publication
(10) Pub. No.: US 2007/0129377 A1
(43) Pub. Date: Jun. 7, 2007

(54) Process for Preparing Substituted Aryl Cycloalkanol Derivatives

(75) Inventors: Alexander V. Gontcharov, Rivervale, NJ (US); Antonia Nikitenko, Tarrytown, NY (US); Jean Schmid, Chester, NY (US); John R. Potoski, West Nyack, NY (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Wyeth, Madison, NJ (US)

(21) Appl. No.: 11/566,415

(22) Filed: Dec. 4, 2006

Related U.S. Application Data
(60) Provisional application No. 60/742,310, filed on Dec. 5, 2005.

(51) Int. Cl. A61K 31/495; C07D 241/04

(52) U.S. Cl. 514/252.12; 544/400

(57) ABSTRACT

Processes are disclosed for preparing substituted aryl cycloalkanol derivatives, particularly chiral substituted aryl cycloalkanol derivatives of the general formula:

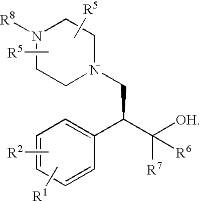

### CROSS REFERENCE TO RELATED APPLICATIONS

This application claims to the benefit of U.S. Provisional Application No. 60/742,310 filed Dec. 5, 2005, the content of which is incorporated herein by reference.

### FIELD OF THE INVENTION

The present invention relates to processes for preparing substituted aryl cycloalkanol derivatives, particularly chiral substituted aryl cycloalkanol derivatives.

### BACKGROUND OF THE INVENTION

Certain substituted aryl cycloalkanol derivatives, such as those disclosed in US-A1-2005/0143579 (the disclosure of which is hereby incorporated herein by reference in its entirety), are useful in preventing and treating conditions ameliorated by monoamine reuptake including, inter alia, vasomotor symptoms (VMS), sexual dysfunction, gastrointestinal and genitourinary disorders, chronic fatigue syndrome, fibromylagia syndrome, nervous system disorders, and combinations thereof, particularly those conditions selected from the group consisting of major depressive disorder, vasomotor symptoms, stress and urge urinary incontinence, fibromyalgia, pain, diabetic neuropathy, and combinations thereof. These compounds were prepared as racemic mixtures. US-A1-2005/0143579 broadly discloses that the final aryl cycloalkanol may be resolved to provide the desired [S]-isomer. More specifically, US-A1-2005/0143579 discloses that the final alkanol or its precursor amide or amine may be resolved by employing either high performance liquid chromatography or supercritical fluid chromatography. Preparation of an enantiomeric aryl cycloalkanol was also described in US-A1-2005/0143579 using a selective enzyme-catalyzed hydrolysis of a racemic intermediate hydroxyacid lower alkyl ester, resulting in the formation of a mixture of the corresponding chiral acid (from the enantioselectively hydrolyzed ester) and chiral ester (from un-hydrolyzed chiral intermediate), which can be more readily separated by conventional means.

There is a ongoing need for more facile and higher yielding processes for preparing substituted aryl cycloalkanol derivatives, particularly chiral substituted aryl cycloalkanol derivatives, useful for, inter alia, preventing and treating conditions ameliorated by monoamine reuptake including, e.g., vasomotor symptoms (VMS), sexual dysfunction, gastrointestinal and genitourinary disorders, chronic fatigue syndrome, fibromylagia syndrome, nervous system disorders, and combinations thereof. The present invention is directed to processes for preparing such substituted aryl cycloalkanol derivatives, particularly chiral substituted aryl cycloalkanol derivatives, for these and other important uses.

### SUMMARY OF THE INVENTION

The present invention is generally directed to processes for preparing substituted aryl cycloalkanol derivatives.

In some embodiments, the present invention is directed to processes for preparing a substituted aryl cycloalkanol compound, comprising the steps of:

contacting a phenylacetic acid of formula I: 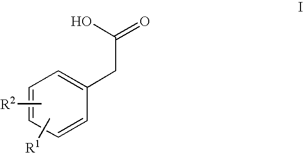
with a ketone of formula II: 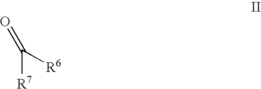
in the presence of a base for a time and under conditions effective to provide an acid compound of formula III: 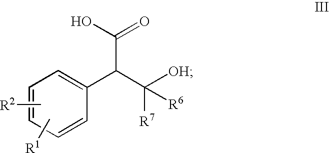
resolving the acid compound of formula III with an acid-resolving chiral amine for a time and under conditions effective to provide an acid compound of formula III*:

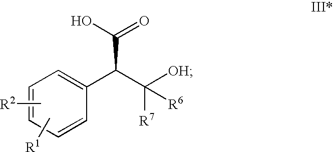

contacting the compound of formula III* with a piperazine compound of formula V: 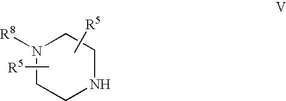
in the presence of a coupling reagent for a time and under conditions effective to provide an amide compound of formula VI*:

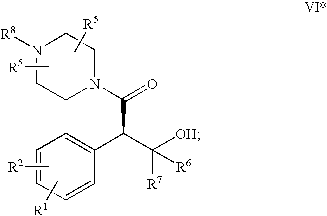

contacting the amide compound of formula VI* with an amide reducing agent for a time and under conditions effective to provide an amine compound of formula VII*: 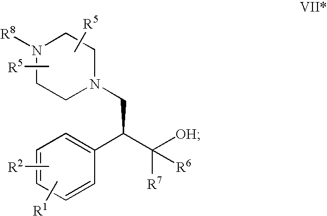
wherein:

R¹ is phenyl, naphthyl, heteroaryl, benzyloxy, phenoxy, naphthyloxy, phenylethoxy, phenoxyethoxy, naphthylmethoxy, naphthylethoxy, phenylcarbonylamino, phenylaminocarbonyl, trifluoromethoxy, nitrile, alkenyl, alkynyl, sulfonyl, sulfonamido, alkanoyl, alkoxycarbonyl, alkylaminocarbonyl, or amino;

where said phenyl, naphthyl, heteroaryl, benzyloxy, phenoxy, naphthyloxy, phenylethoxy, phenoxyethoxy, naphthylmethoxy, naphthylethoxy, phenylcarbonylamino, and phenylaminocarbonyl are optionally substituted with one or more substituents as defined for R²;

R² is H, or one or two substituents, the same or different selected from the group consisting of OH, alkyl, alkoxy, halo, trifluoromethyl, alkanoyloxy, methylenedioxy, trifluoromethoxy, nitrile, nitro, alkenyl, alkynyl, sulfonyl, and sulfonamido;

each R⁵ is independently H, (C₁-C₆)alkyl, or trifluoromethyl;

R⁶ and R⁷ are, independently, (C₁-C₆)alkyl optionally substituted with R⁵ or OH, or (C₃-C₆)cycloalkyl optionally substituted with R⁵ or OH;

or R⁶ and R⁷, taken together with the carbon atom to which they are attached, form a 4- to 8-membered cycloalkyl ring optionally substituted with R⁵ or OH,

or R⁶ and R⁷, taken together with the carbon atom to which they are attached, form a 4- to 8-membered cycloalkyl ring fused to a 4- to 6-membered cycloalkyl ring, wherein either or both of said cycloalkyl rings is optionally substituted with R⁵ or OH,

where any carbon atom of said R⁶ and R⁷ may be optionally replaced with N, S, or O;

R⁸ is H, (C₁-C₆)alkyl, hydroxy(C₁-C₆)alkyl, benzyl (optionally substituted with benzyloxy or phenyloxy), naphthylmethyl (optionally substituted with one or more R¹), phenyl(C₂-C₆)alkyl (optionally substituted with one or more R¹), heteroarylmethyl (optionally substituted with R¹), cycloalkyl, cycloalkenyl, cycloalkylmethyl (where any carbon atom can be optionally replaced with N, S, or O and where said cycloalkylmethyl can be optionally substituted with OH, CF₃, halo, alkoxy, alkyl, benzyloxy, or alkanoyloxy), cycloalkenylmethyl (where any carbon atom can be optionally replaced with N, S, or O and where said cycloalkenylmethyl can be optionally substituted with OH, CF₃, halo, alkoxy, alkyl, benzyloxy, or alkanoyloxy);

or R⁵ and R⁸, taken together with the nitrogen and carbon atoms through which they are connected, form a 4- to 8-membered heterocycloalkyl ring, more preferably a 5- to 6-membered, still more preferably a 6-membered ring; said heterocycloalkyl ring optionally substituted with R⁵.

In some embodiments, the present invention is directed to processes for preparing compounds of formula VI* or VII* wherein the compounds of formula VI*, VII*, and their precursor compounds of formula V, are substituted with R⁵.

In other embodiments, the present invention is directed to processes for preparing a substituted aryl cycloalkanol compound, comprising the steps of:

contacting a phenylacetic acid of formula I: 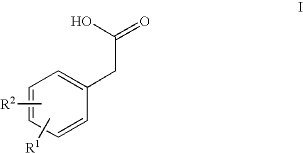
with thionyl chloride and a piperazine compound of formula V:

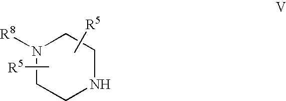

for a time and under conditions effective to provide an amide compound of formula VII:

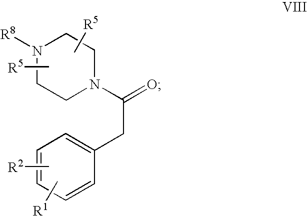

contacting the amide compound of formula VIII with a ketone of formula II: 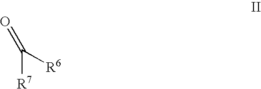
in the presence of a base for a time and under conditions effective to provide an amide compound of formula VI:

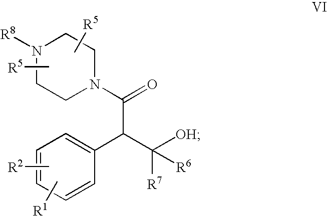

contacting the amide compound of formula VI with an amide reducing agent for a time and under conditions effective to provide an amine compound of formula VII:

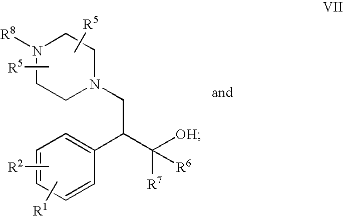

resolving the amine compound of formula VII with an amine-resolving chiral acid for a time and under conditions effective to provide an amine compound of formula VII*:

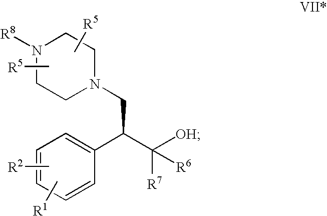

wherein R¹, R², R⁵, R⁶, R⁷ and R⁸ are as defined herein.

In some embodiments, the present invention is directed to processes for preparing compounds of formula VI* or VII* wherein the compounds of formula VI*, VII*, and their precursor compounds of formula V, are substituted with R⁵.

In certain embodiments, the present invention is directed to processes for preparing a substituted aryl cycloalkanol compound, comprising the steps of:

contacting a benzaldehyde of formula IX:

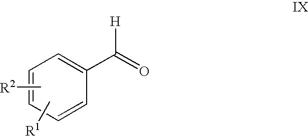

with carbon tetrabromide and a triaryl phosphine for a time and under conditions effective to provide a dibromoalkene compound of formula X:

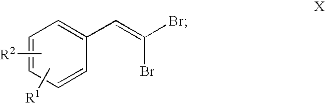

contacting the dibromoalkene compound of formula X with a piperazine compound of formula V:

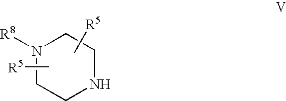

for a time and under conditions effective to provide an amide compound of formula VII:

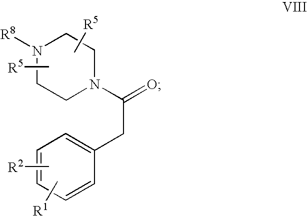

contacting the amide compound of formula VIII with a ketone of formula II: 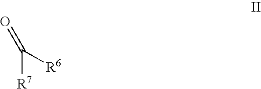
in the presence of a base for a time and under conditions effective to provide an amide compound of formula VI:

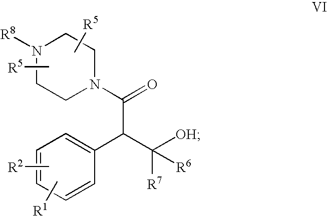

contacting the amide compound of formula VI with an amide reducing agent for a time and under conditions effective to provide an amine compound of formula VII: 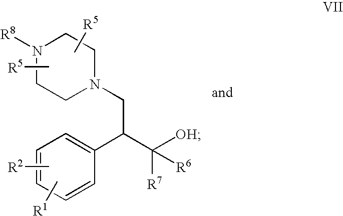
resolving the amine compound of formula VII with an amine-resolving chiral acid for a time and under conditions effective to provide an amine compound of formula VII*

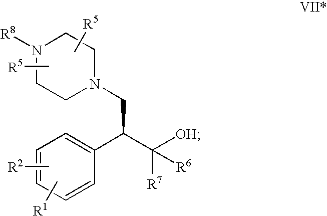

wherein R¹, R², R⁵, R⁶, R⁷ and R⁸ are as defined herein.

In some embodiments, the present invention is directed to processes for preparing compounds of formula VI* or VII* wherein the compounds of formula VI*, VII*, and their precursor compounds of formula V, are substituted with R⁵.

In still other embodiments, the present invention is directed to processes for preparing a non-racemic substituted aryl cycloalkanol compound, comprising the steps of:

resolving the acid compound of formula III:

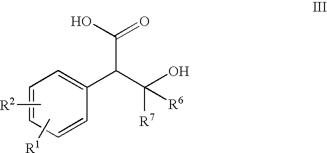

with an acid-resolving chiral amine for a time and under conditions effective to provide an acid compound of formula III*:

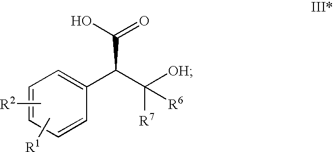

wherein R¹, R², R⁶ and R⁷ are as defined herein.

### DETAILED DESCRIPTION OF ILLUSTRATIVE EMBODIMENTS

The present invention is directed to processes for preparing substituted aryl cycloalkanol derivatives, and more particularly, to processes for preparing substituted aryl cycloalkanol derivatives useful, alone, or in compositions, for the prevention and treatment of conditions ameliorated by monoamine reuptake including, inter alia, vasomotor symptoms (VMS), sexual dysfunction, gastrointestinal and genitourinary disorders, chronic fatigue syndrome, fibromylagia syndrome, nervous system disorders, and combinations thereof, particularly those conditions selected from the group consisting of major depressive disorder, vasomotor symptoms, stress and urge urinary incontinence, fibromyalgia, pain, diabetic neuropathy, and combinations thereof.

The following definitions are provided for the full understanding of terms and abbreviations used in this specification.

As used herein and in the appended claims, the singular forms “a,” “an,” and “the” include the plural reference unless the context clearly indicates otherwise. Thus, for example, a reference to “an antagonist” includes a plurality of such antagonists, and a reference to “a compound” is a reference to one or more compounds and equivalents thereof known to those skilled in the art, and so forth.

The abbreviations in the specification correspond to units of measure, techniques, properties, or compounds as follows: “min” means minutes, “h” means hour(s), “pL” means microliter(s), “mL” means milliliter(s), “mM” means millimolar, “M” means molar, “mmole” means millimole(s), “cm” means centimeters, “SEM” means standard error of the mean and “IU” means International Units. “Δ° C.” and A “ED₅₀ value” means dose which results in 50% alleviation of the observed condition or effect (50% mean maximum endpoint).

“Norepinephrine reuptake inhibitor” is abbreviated NRI.

“Serotonin reuptake inhibitor” is abbreviated SRI.

“Norepinephrine” is abbreviated NE.

“Serotonin is abbreviated 5-HT.

The terms “component,” “composition of compounds,” “compound,” “drug,” or “pharmacologically active agent” or “active agent” or “medicament” are used interchangeably herein to refer to a compound or compounds or composition of matter which, when administered to a subject (human or animal) induces a desired pharmacological and/or physiologic effect by local and/or systemic action.

The terms “component”, “drug” or “pharmacologically active agent” or “active agent” or “medicament” are used interchangeably herein to refer to a compound or compounds or composition of matter which, when administered to an organism (human or animal) induces a desired pharmacologic and/or physiologic effect by local and/or systemic action.

The term “modulation” refers to the capacity to either enhance or inhibit a functional property of a biological activity or process, for example, receptor binding or signaling activity. Such enhancement or inhibition may be contingent on the occurrence of a specific event, such as activation of a signal transduction pathway and/or may be manifest only in particular cell types. The modulator is intended to comprise any compound, e.g., antibody, small molecule, peptide, oligopeptide, polypeptide, or protein, preferably small molecule, or peptide.

As used herein, the term “inhibitor” refers to any agent that inhibits, suppresses, represses, or decreases a specific activity, such as serotonin reuptake activity or the norepinephrine reuptake activity.

The term “inhibitor,” as used herein, is intended to comprise any compound, e.g., antibody, small molecule, peptide, oligopeptide, polypeptide, or protein, preferably small molecule or peptide, that exhibits a partial, complete, competitive and/or inhibitory effect on mammalian, preferably the human norepinephrine reuptake or both serotonin reuptake and the norepinephrine reuptake, thus diminishing or blocking, preferably diminishing, some or all of the biological effects of endogenous norepinephrine reuptake or of both serotonin reuptake and the norepinephrine reuptake.

Within the present invention, the compounds of formula I may be prepared in the form of pharmaceutically acceptable salts. As used herein, the term “pharmaceutically acceptable salts” refers to salts prepared from pharmaceutically acceptable non-toxic acids, including inorganic salts, and organic salts. Suitable non-organic salts include inorganic and organic acids such as acetic, benzenesulfonic, benzoic, camphorsulfonic, citric, ethanesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferably is the hydrochloride salt.

The term “alkyl,” as used herein, refers to an aliphatic hydrocarbon chain of 1 to about 20 carbon atoms, preferably 1 to 10 carbon atoms, more preferably, 1 to 6 carbon atoms, and even more preferably, 1 to 4 carbon atoms and includes straight and branched chains such as methyl, ethyl, n-propyl, isopropyl, n-butyl, isobutyl, sec-butyl, t-butyl, n-pentyl, isopentyl, neo-pentyl, n-hexyl, and isohexyl. Lower alkyl refers to alkyl having 1 to 4 carbon atoms.

The term “alkylenyl,” as used herein, refers to a bivalent aliphatic hydrocarbon chain of 1 to about 20 carbon atoms, preferably 1 to 10 carbon atoms, more preferably, 1 to 6 carbon atoms, and even more preferably, 1 to 4 carbon atoms and includes straight and branched chains such as methylenyl,

The term “alkoxy,” as used herein, refers to the group R—O— where R is an alkyl group of 1 to 6 carbon atoms.

The term “alkoxycarbonyl,” as used herein, refers to the group R—O—C(═O)— where R is an alkyl group of 1 to 6 carbon atoms.

The term “alkanoyl,” as used herein, refers to the group R—C(═O)— where R is an alkyl group of 1 to 6 carbon atoms.

The term “alkanoyloxy,” as used herein, refers to the group R—C(═O)—O— where R is an alkyl group of 1 to 6 carbon atoms.

The term “alkylaminocarbonyl,” as used herein, refers to the group R—NH— C(═O)— where R is an alkyl group of 1 to 6 carbon atoms.

The term “alkylcarbonylamino,” as used herein, refers to the group R— C(═O)—NH where R is an alkyl group of 1 to 6 carbon atoms.

The term “alkenyl” or “olefinic,” as used herein, refers to an alkyl group of at least two carbon atoms, e.g., 2 to 20 carbon atoms, having one or more double bonds, wherein alkyl is as defined herein. Alkenyl groups can be optionally substituted.

The term “alkynyl,” as used herein, refers to an alkyl group of at least two carbon atoms, e.g., 2 to 20 carbon atoms, having one or more triple bonds, wherein alkyl is as defined herein. Alkynyl groups can be optionally substituted.

The term “aryl” as used herein, refers to an optionally substituted, mono-, di-, tri-, or other multicyclic aromatic ring system having from about 5 to about 50 carbon atoms (and all combinations and subcombinations of ranges and specific numbers of carbon atoms therein), with from about 6 to about 10 carbons being preferred. Non-limiting examples include, for example, phenyl, naphthyl, anthracenyl, and phenanthrenyl.

The term “heteroaryl,” as used herein, refers to an optionally substituted, mono-, di-, tri-, or other multicyclic aromatic ring system that includes at least one, and preferably from 1 to about 4 heteroatom ring members selected from sulfur, oxygen and nitrogen. Heteroaryl groups can have, for example, from about 4 to about 50 ring atoms (and all combinations and subcombinations of ranges and specific numbers of atoms therein, e.g., 5 to 20), with from about 5 to about 10 ring atoms being preferred. Non-limiting examples of heteroaryl groups include, for example, pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl.

The term “heteroarylmethyl,” as used herein, refers to the group R—CH₂— where R is a heteroaryl group, as defined herein.

The term “cycloalkyl,” as used herein, refers to an optionally substituted, alkyl group having one or more rings in their structures having from 3 to about 20 carbon atoms (and all combinations and subcombinations of ranges and specific numbers of carbon atoms therein), with from 3 to about 10 carbon atoms being preferred. Multi-ring structures may be bridged or fused ring structures. Groups include, but are not limited to, cyclopropyl, cyclobutyl, cyclopentyl, cyclohexyl, cyclooctyl, 2-[4-isopropyl-1-methyl-7-oxa-bicyclo[2.2.1 ]heptanyl], 2-[1,2,3,4-tetrahydro-naphthalenyl], and adamantyl.

The term “cycloalkylmethyl,” as used herein, refers to the group R—CH₂— where R is a cycloalkyl group, as defined herein.

The term “cycloalkenyl,” as used herein, refers to an optionally substituted, alkene group having one or more rings in their structures having from 3 to about 20 carbon atoms (and all combinations and subcombinations of ranges and specific numbers of carbon atoms therein), with from 3 to about 10 carbon atoms being preferred. Multi-ring structures may be bridged or fused ring structures. Groups include, but are not limited to, cyclopropenyl, cyclobutenyl, cyclopentenyl, cyclohexenyl, and cyclooctenyl.

The term “cycloalkenylmethyl,” as used herein, refers to the group R—CH₂— where R is a cycloalkenyl group, as defined herein.

The term “carbodiimide,” as used herein, refers to a compound of formula R—N═C═N—R, wherein each R is independently an optionally substituted cyclic or alicyclic aliphatic or aromatic hydrocarbon.

The term “sulfonamido,” as used herein, refers to a moiety containing the group —S(O)₂—NR—, where R is H or alkyl, as defined herein.

The term “sulfonyl,” as used herein, refers to a moiety containing the group —S(O)₂— and —S(O)₂—R—, where R is alkylenyl, as defined herein, including alkylsulfonyl.

The term “halo” or “halogen,” as used herein, refers to chloro, bromo, fluoro, and iodo.

As used herein, the term “contacting” refers to the bringing together of compounds to within distances that allow for intermolecular interactions and chemical transformations accompanying such interactions. The term “contacting” includes chemical reactions between two or more reactants. Often, contacting compounds are in solution phase. To optimize yields reactions are suitably carried out for a time and under conditions effective to provide the desired product.

As used herein, the term “resolving” refers to any process of enhancing or enriching in a product the level of one enantiomer over its antipode from any mixture of the two enantiomers. Such mixtures include those where the enantiomers are present in equal amounts (racemates) or unequal amounts (those mixtures having an enantiomeric excess or one or the other of the enantiomers.

It is believed the chemical formulas and names used herein correctly and accurately reflect the underlying chemical compounds. However, the nature and value of the present invention does not depend upon the theoretical correctness of these formulae, in whole or in part. Thus it is understood that the formulas used herein, as well as the chemical names attributed to the correspondingly indicated compounds, are not intended to limit the invention in any way, including restricting it to any specific tautomeric form or to any specific optical or geometric isomer.

When ranges are used herein for physical properties, such as molecular weight, or chemical properties, such as chemical formulae, all combinations and subcombinations of ranges specific embodiments therein are intended to be included.

When any variable occurs more than one time in any constituent or in any formula, its definition in each occurrence is independent of its definition at every other occurrence. Combinations of substituents and/or variables are permissible only if such combinations result in stable compounds.

In compounds where a carbon atom may be replaced by a heteroatom, such as a N, S, or O, each of such replacement groups may be substituted in the same manner as the carbon atom, if such substitution is technically feasible and does not violate valence or form an unstable species. Thus, for example, if any carbon ring atom may be substituted by —OH or R⁵, then the carbon atom (if replaced) may be NH, NR⁵, NOH, S, or O, even if such substitution is not explicitly stated.

In certain embodiments, the present invention is directed to processes for preparing a substituted aryl cycloalkanol compound, comprising the steps of:

reacting a phenylacetic acid of formula I: 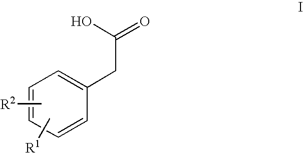
with a ketone of formula II: 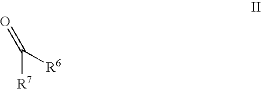
in the presence of a base to provide an acid compound of formula III:

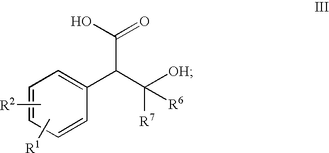

resolving the acid compound of formula III with an acid-resolving chiral amine to provide an acid compound of formula III*:

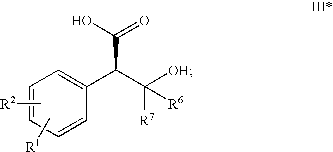

reacting the compound of formula III* with a piperazine compound of formula V: 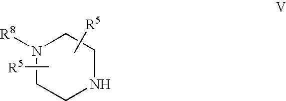
in the presence of a coupling reagent to provide an amide compound of formula VI*:

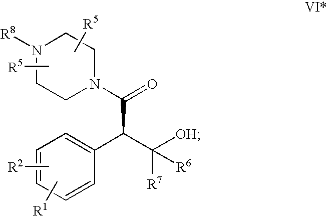

reacting the amide compound of formula VI* with an amide reducing agent to provide an amine compound of formula VII*:

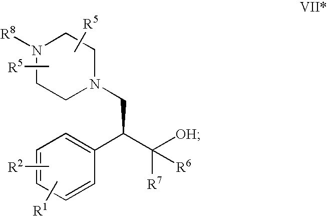

wherein R¹, R², R⁵, R⁶, R⁷ and R⁸ are as defined herein.

In certain preferred embodiments, the processes further comprise the step of contacting the compound of formula VIII*: 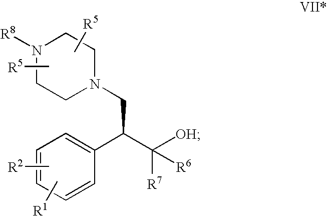
with hydrochloric acid for a time and under conditions effective to form the hydrochloride salt of the compound of formula VII*, more preferably wherein the hydrochloride salt is the dihydrochloride salt of the compound of formula VII*. Even more preferably the process further comprises the step of re-crystallizing of the hydrochloride salt of the compound of formula VII*, wherein the hydrochloride salt is recrystallized from a solvent comprising an alcohol or alcohol-ether mixture, yet still more preferably wherein the alcohol-ether mixture comprises methyl tertiary butyl ether and methanol.

In certain preferred embodiments, R¹ and R² are each independently H or trifluoromethoxy, more preferably at least one of R¹ and R² is trifluoromethoxy, yet more preferably R¹ is trifluoromethoxy, still more preferably R¹ is trifluoromethoxy and R² is H.

In other preferred embodiments, the compound of formula I is a phenylacetic acid substituted with one or two trifluoromethoxy groups, more preferably one trifluoromethoxy group, still more preferably the compound of formula I is trifluoromethoxyphenylacetic acid, yet more preferably meta-(trifluoromethoxy)phenylacetic acid.

In some preferred embodiments, R⁵ in the piperazine ring is H or (C₁-C₆)alkyl. When R⁵ is (C₁-C₆)alkyl, it is preferably (C₁-C₃)alkyl, more preferably methyl.

In other preferred embodiments, R⁶ and R⁷, taken together with the carbon atom to which they are attached form a ring of 4 to 8 carbon atoms, more preferably 5 to 6 carbon atoms, still more preferably 6 carbon atoms.

In certain preferred embodiments, the compound of formula II is cyclohexanone.

In some preferred embodiments, the compound of formula III is (1-hydroxy-cyclohex-1-yl)-(3-trifluoromethoxyphenyl)-acetic acid.

In certain preferred embodiments, the compound of formula III* is (R)—(1-hydroxy-cyclohex-1-yl)-(3-trifluoromethoxyphenyl)-acetic acid.

In some preferred embodiments, R⁸ is H.

In other preferred embodiments, the compound of formula V is dimethylpiperazine, more preferably 2,6-dimethylpiperazine, still more preferably [2S*,6R*]-dimethylpiperazine.

In certain preferred embodiments, the compound of formula VI* is 1-(3,5-dimethylpiperazin-1-yl)-2R-(1-hydroxycyclohex-1-yl)-2-(3-trifluoromethoxyphenyl) ethanone, more preferably 1-(3S*,5R*-dimethylpiperazin-1-yl)-2R-(1-hydroxycyclohex-1-yl)-2-(3-(trifluoromethoxy)phenyl)ethanone.

In some preferred embodiments, the compound of formula VII* is 1S-[2-(3,5-dimethylpiperazin-1-yl)-1-(3-trifluoromethoxyphenyl)ethyl]cyclohexanol, with 1S-[2-(3S*,5R*-dimethylpiperazin-1-yl)-1-(3-(trifluoromethoxy)phenyl)ethyl]cyclohexanol being more preferred.

In certain preferred embodiments of the above mentioned processes, the base is MH, MNR⁹R⁹, alkyl lithium, or aryl lithium, or any combination thereof, wherein M is sodium, potassium or lithium, and each R⁹ is independently H, alkyl, Si(alkyl)₃.

In certain preferred embodiments of the above mentioned processes, the acid-resolving chiral amine is (S)-methylbenzylamine, (R)-methylbenzylamine, D-(+)-aminobutanol, (+)-dehydroabiethylamine, (−)-ephedrine, (−)-pseudoephedrine, (−)-norephedrine, (−)-cinchonidine, brucine, (+)-benzylphenethylamine, (−)-benzylphenethylamine, (−)-(alpha-phenylpropyl)amine, (+)-2-aminoethanol, or quinidine, or any combination thereof. More preferably, the acid-resolving chiral amine is (S)-methylbenzylamine, (R)-methylbenzylamine, (+)-dehydroabiethylamine, (+)-benzylphenethylamine, or (−)-benzylphenethylamine, or any combination thereof. In some embodiments of the invention, the level of the (S)-isomer is preferentially enriched by resolving. When the (S)-isomer is the desired isomer, the resolving is preferentially conducted with an acid-resolving chiral amine selected from the group consisting of (S)-methylbenzylamine, (+)-dehydroabiethylamine, or (+)-benzylphenethylamine, or any combination thereof. In other embodiments of the invention, the level of the (R)-isomer is preferentially enriched by resolving. When the (R)-isomer is the desired isomer, the resolving is preferentially conducted with an acid-resolving chiral amine selected from the group consisting of (R)-methylbenzylamine or (−)-benzylphenethylamine, or any combination thereof.

In some embodiments, the enantiomeric excess for the compound of formula III* after the resolving of the compound of formula III is at least about 20%, more preferably, at least about 40%, still more preferably at least about 60%, yet more preferably about 80%, even more preferably, at least about 90% of the desired chiral isomer, and yet even more preferably at least 95% of the desired chiral isomer. For example, after resolving the compound of formula III the (S)-isomer of formula III* is in enantiomeric excess of at least about 20%.

In other preferred embodiments of the above mentioned process, the coupling reagent is (benzotriazol-1-yloxy)tris(dimethylamino)phosphonium hexafluorophosphate (BOP), (benzotriazol-1-yloxy)tripyrrolidinophosphonium hexafluorophosphate (PyBOP), or a N,N-dialkyl, N,N-diaryl, or N-aryl-N-alkyl carbodiimide (with or without optional 1-hydroxybenzotriazole). Other suitable coupling reagents can be found as described by Benz. G., “Synthesis of Amides and Related Compounds”, Chapter 2.3, Comprehensive Organic Synthesis, Volume 6, pages 381-417, Trost, B. M., ed., Pergammon Press, 1^{st} Ed., NY (1991), or by Bailey, P. D. et al., “Amides”, Chapter 5.06. “Comprehensive Organic Functional Group Transformations, Volume 5, pages 257-307, Katritsky, A. R., ed., Pergammon Press, 1^{st} Ed., NY (1995), the disclosures of which are hereby incorporated herein by reference, in their entireties.

In still other preferred embodiments of the above mentioned process, the amide reducing agent is borane, bis(2-methoxyethoxy)aluminum hydride, allane, AlH₂Cl or other chloroaluminum hydrides, lithium aluminum hydride, DIBAL, or a mixture thereof.

In other embodiments, the invention is directed to processes for preparing a substituted aryl cycloalkanol compound, comprising the steps of:

reacting a phenylacetic acid of formula I:

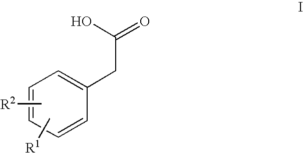

with a piperazine compound of formula V: 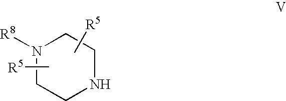
in the presence of a coupling agent to provide an amide compound of formula VIII:

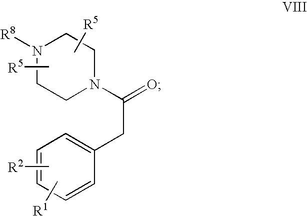

reacting the amide compound of formula VIII with a ketone of formula II:

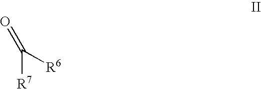

in the presence of a base to provide an amide compound of formula VI:

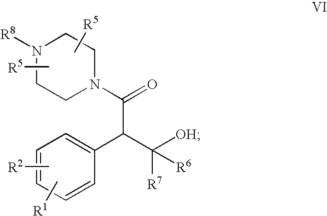

reacting the amide compound of formula VI with an amide reducing agent to provide an amine compound of formula VII:

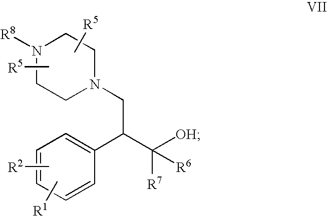

resolving the amine compound of formula VII with an amine-resolving chiral acid to provide an amine compound of formula VII*: 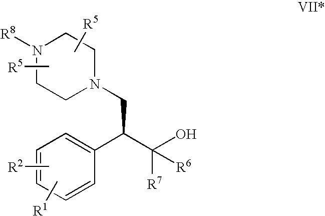
wherein:

R¹ is phenyl, naphthyl, heteroaryl, benzyloxy, phenoxy, naphthyloxy, phenylethoxy, phenoxyethoxy, naphthylmethoxy, naphthylethoxy, phenylcarbonylamino, phenylaminocarbonyl, trifluoromethoxy, nitrile, alkenyl, alkynyl, sulfonyl, sulfonamido, alkanoyl, alkoxycarbonyl, alkylaminocarbonyl, or amino;

where said phenyl, naphthyl, heteroaryl, benzyloxy, phenoxy, naphthyloxy, phenylethoxy, phenoxyethoxy, naphthylmethoxy, naphthylethoxy, phenylcarbonylamino, and phenylaminocarbonyl are optionally substituted with one or more R²;

R² is H, or one or two substituents, the same or different selected from the group consisting of OH, alkyl, alkoxy, halo, trifluoromethyl, alkanoyloxy, methylenedioxy, trifluoromethoxy, nitrile, nitro, alkenyl, alkynyl, sulfonyl, and sulfonamido;

each R⁵ is independently H, (C₁-C₆)alkyl, or trifluoromethyl;

R⁶ and R⁷ are, independently, (C₁-C₆)alkyl optionally substituted with R⁵ or OH, or (C₃-C₆)cycloalkyl optionally substituted with R⁵ or OH;

or R⁶ and R⁷, taken together with the carbon atom to which they are attached, form a 4- to 8-membered cycloalkyl ring optionally substituted with R⁵ or OH,

or R⁶ and R⁷, taken together with the carbon atom to which they are attached, form a 4- to 8-membered cycloalkyl ring fused to a 4- to 6-membered cycloalkyl ring, wherein either or both of said cycloalkyl rings is optionally substituted with R⁵ or OH,

where any carbon atom of said R⁶ and R⁷ may be optionally replaced with N, S, or O;

R⁸ is H, (C₁-C₆)alkyl, hydroxy(C₁-C₆)alkyl, benzyl (optionally substituted with benzyloxy or phenyloxy), naphthylmethyl (optionally substituted with one or more R¹), phenyl(C₂-C₆)alkyl (optionally substituted with one or more R¹), heteroarylmethyl (optionally substituted with R¹), cycloalkyl, cycloalkenyl, cycloalkylmethyl (where any carbon atom can be optionally replaced with N, S, or O and where said cycloalkylmethyl can be optionally substituted with OH, CF₃, halo, alkoxy, alkyl, benzyloxy, or alkanoyloxy), cycloalkenylmethyl (where any carbon atom can be optionally replaced with N, S, or O and where said cycloalkenylmethyl can be optionally substituted with OH, CF₃, halo, alkoxy, alkyl, benzyloxy, or alkanoyloxy);

or R⁵ and R⁸, taken together with the nitrogen and carbon atoms through which they are connected, form a 4- to 8-membered heterocycloalkyl ring, more preferably a 5- to 6-membered, still more preferably a 6-membered ring; said heterocycloalkyl ring optionally substituted with R⁵.

In certain preferred embodiments, wherein the phenylacetic acid of formula I:

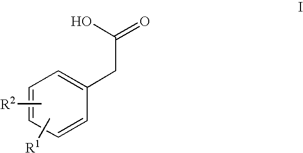

is coupled with a piperazine compound of formula V: 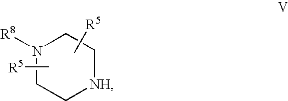
the coupling agent employed may be any reagent which converts an organic acid to the corresponding acid chloride, such as for example, thionyl chloride. Alternatively, the coupling reagent may an N,N-dialkyl, N,N-diaryl, or N-alkyl-N-aryl-carbodiimide, alone or in conjunction with a catalyst such as 1-hydroxybenzotriazole. In other preferred embodiments, the coupling agent may be (benzotriazol-1-yloxy)tris(dimethylamino)phosphonium hexafluorophosphate (BOP), (benzotriazol-1-yloxy)tripyrrolidinophosphonium hexafluorophosphate (PyBOP), or other such compounds mentioned in this application or known to one of ordinary skill in the art as a reagent that is effective in the coupling of amines with carboxylic acids to form the corresponding amides such as, for example, those disclosed in Benz. G., “Synthesis of Amides and Related Compounds”, Chapter 2.3, Comprehensive Organic Synthesis, Volume 6, pages 381-417, Trost, B. M., ed., Pergammon Press, 1^{st} Ed., NY (1991), or by Bailey, P. D. et al., “Amides”, Chapter 5.06. “Comprehensive Organic Functional Group Transformations, Volume 5, pages 257-307, Katritsky, A. R., ed., Pergammon Press, 1^{st} Ed., NY (1995), the disclosures of which are hereby incorporated herein by reference, in their entireties.

In still other embodiments, the invention is directed to processes for preparing a substituted aryl cycloalkanol compound, comprising the steps of:

reacting a benzaldehyde of formula IX: 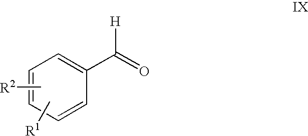
with carbon tetrabromide and a triaryl phosphine o provide a dibromoalkene compound of formula X:

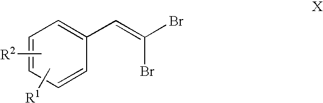

reacting the dibromoalkene compound of formula X with a piperazine compound of formula V: 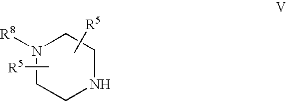
to provide an amide compound of formula VIII:

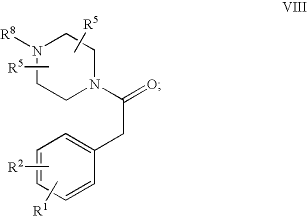

reacting the amide compound of formula VIII with a ketone of formula II: 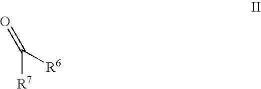
in the presence of a base to provide an amide compound of formula VI:

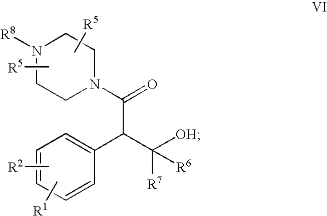

reacting the amide compound of formula VI with an amide reducing agent to provide an amine compound of formula VII: 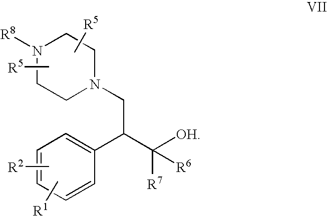
resolving the amine compound of formula VII with an amine-resolving chiral acid to provide an amine compound of formula VII*

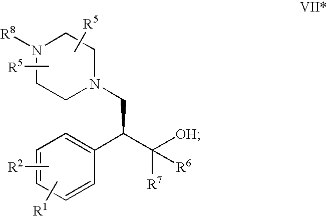

wherein R¹, R², R³, R⁵, R⁶, R⁷ and R⁸ are as defined herein

In still other embodiments, the present invention is directed to processes for preparing a non-racemic substituted aryl cycloalkanol compound, comprising the steps of:

resolving the acid compound of formula III:

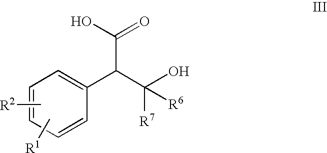

with an acid-resolving chiral amine to provide an acid compound of formula III*:

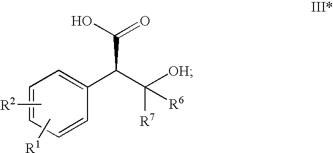

wherein R¹, R², R⁶ and R⁷ are as defined herein or a pharmaceutically acceptable salt thereof.

In certain preferred embodiments of the above mentioned processes, the acid-resolving chiral amine is (S)-methylbenzylamine, (R)-methylbenzylamine, D-(+)-aminobutanol, (+)-dehydroabiethylamine, (−)-ephedrine, (−)-pseudoephedrine, (−)-norephedrine, (−)-cinchonidine, brucine, (+)-benzylphenethylamine, (−)-benzylphenethylamine, (−)-(alpha-phenylpropyl)amine, (+)-2-aminoethanol, or quinidine, or any combination thereof. More preferably, the acid-resolving chiral amine is (S)-methylbenzylamine, (R)-methylbenzylamine, (+)-dehydroabiethylamine, (+)-benzylphenethylamine, or (−)-benzylphenethylamine, or any combination thereof. In some embodiments of the invention, the level of the (S)-isomer is preferentially enriched by resolving. When the (S)-isomer is the desired isomer, the resolving is preferentially conducted with an acid-resolving chiral amine selected from the group consisting of (S)-methylbenzylamine, (+)-dehydroabiethylamine, or (+)-benzylphenethylamine, or any combination thereof. In other embodiments of the invention, the level of the (R)-isomer is preferentially enriched by resolving. When the (R)-isomer is the desired isomer, the resolving is preferentially conducted with an acid-resolving chiral amine selected from the group consisting of (R)-methylbenzylamine or (−)-benzylphenethylamine, or any combination thereof.

In some embodiments of the above mentioned process, the enantiomeric excess for the compound of formula III* after the resolving of the compound of formula III is at least about 20%, more preferably, at least about 40%, still more preferably at least about 60%, yet more preferably about 80%, even more preferably, at least about 90% of the desired chiral isomer, and yet even more preferably at least 95% of the desired chiral isomer.

In certain preferred embodiments of the above mentioned process, the resolution is carried out in the presence of a resolution solvent, preferably an inert solvent. Non-limiting examples include hydrocarbon and nitrile solvents, such as hexane, hexanes, and acetonitrile, and mixtures thereof. In some preferred embodiments, the acid and amine may be individually dissolved in an alcoholic or other compatible solvent prior to contact with each other. Non-limiting examples include C₁-C₃ alcohols, with methanol being more preferred. In other preferred embodiments, the alcoholic or other compatible solvent is evaporated before the resolution solvent is added. The temperature during the resolution step is not critical, but by way of general guidance, the mixture may be warmed initially to enhance the solubility of the compound of formula III and the selected acid-resolving chiral amine in each other and/or the solvent. Any optional heat added may then be removed and the resolution may be carried out at a temperature within the range of about 0° C. to about 25° C. In some preferred embodiments, the crystals formed in the resolution step may be isolated and further resolved by repeating the process. The ratio of acid the acid resolving amine is not critical, but for reasons of efficiency, a typical equivalent range of amine to desired enantiomeric acid is from about 0.5:1 to about 1.5:1, more preferably about 0.75:1 to about 1.25, with from about 1:1 to about 1.2:1 being even more preferred.

In any of the above noted processes, the following preferred embodiments may apply.

In certain preferred embodiments,

R¹ is phenyl, naphthyl, heteroaryl, benzyloxy, phenoxy, naphthyloxy, phenylethoxy, phenoxyethoxy, naphthylmethoxy, naphthylethoxy, phenylcarbonylamino, phenylaminocarbonyl, trifluoromethoxy, nitrile, alkenyl, alkynyl, sulfonyl, sulfonamido, alkanoyl, alkoxycarbonyl, alkylaminocarbonyl, or amino;

where said phenyl, naphthyl, heteroaryl, benzyloxy, phenoxy, naphthyloxy, phenylethoxy, phenoxyethoxy, naphthylmethoxy, naphthylethoxy, phenylcarbonylamino, and phenylaminocarbonyl are optionally substituted with one or more substituents as defined for R²;

R² is H, OH, alkyl, alkoxy, halo, trifluoromethyl, alkanoyloxy, methylenedioxy, trifluoromethoxy, nitrile, nitro, alkenyl, alkynyl, sulfonyl, or sulfonamido;

each R⁵ is independently H, (C₁-C₆)alkyl or trifluoromethyl;

R⁶ and R⁷ taken together with the carbon atom to which they are attached form a ring of 4 to 8 carbon atoms; and

R⁸ is H, (C₁-C₆)alkyl, hydroxy(C₁-C₆)alkyl, benzyl (optionally substituted with benzyloxy or phenyloxy), naphthylmethyl (optionally substituted with one or more R¹), phenyl(C₂-C₆)alkyl (optionally substituted with one or more R¹), heteroarylmethyl (optionally substituted with R¹), cycloalkyl, cycloalkenyl, cycloalkylmethyl (where any carbon atom can be optionally replaced with N, S, or O and where said cycloalkylmethyl can be optionally substituted with OH, CF₃, halo, alkoxy, alkyl, benzyloxy, or alkanoyloxy), cycloalkenylmethyl (where any carbon atom can be optionally replaced with N, S, or O and where said cycloalkenylmethyl can be optionally substituted with OH, CF₃, halo, alkoxy, alkyl, benzyloxy, or alkanoyloxy);

R⁵ and R⁸, taken together with the nitrogen and carbon atoms through which they are connected, form a 4- to 8-membered heterocycloalkyl ring, more preferably a 5- to 6-membered, still more preferably a 6-membered ring; said heterocycloalkyl ring optionally substituted with R⁵.

In certain preferred embodiments,

R¹ is phenyl, naphthyl, heteroaryl, benzyloxy, phenoxy, naphthyloxy, phenylethoxy, phenoxyethoxy, naphthylmethoxy, or naphthylethoxy;

where said phenyl, naphthyl, heteroaryl, benzyloxy, phenoxy, naphthyloxy, phenylethoxy, phenoxyethoxy, naphthylmethoxy, and naphthylethoxy are optionally substituted with one or more R²;

R² is H, OH, alkyl, alkoxy, halo, trifluoromethyl, alkanoyloxy, methylenedioxy, trifluoromethoxy, nitrile, nitro, alkenyl, alkynyl, sulfonyl, or sulfonamido;

each R⁵ is independently H, (C₁-C₆)alkyl or trifluoromethyl;

R⁶ and R⁷ taken together with the carbon atom to which they are attached form a ring of 4 to 8 carbon atoms; and

R⁸ is H, (C₁-C₆)alkyl, hydroxy(C₁-C₆)alkyl, benzyl (optionally substituted with benzyloxy or phenyloxy), naphthylmethyl (optionally substituted with one or more R¹), phenyl(C₂-C₆)alkyl (optionally substituted with one or more R¹), heteroarylmethyl (optionally substituted with R¹), cycloalkyl, cycloalkenyl, cycloalkylmethyl (where any carbon atom can be optionally replaced with N, S, or O and where said cycloalkylmethyl can be optionally substituted with OH, CF₃, halo, alkoxy, alkyl, benzyloxy, or alkanoyloxy), cycloalkenylmethyl (where any carbon atom can be optionally replaced with N, S, or O and where said cycloalkenylmethyl can be optionally substituted with OH, CF₃, halo, alkoxy, alkyl, benzyloxy, or alkanoyloxy).

In certain preferred embodiments,

R¹ is trifluoromethoxy, nitrile, alkenyl, or alkynyl;

R² is H, OH, alkyl, alkoxy, halo, or trifluoromethyl;

each R⁵ is independently H, (C₁-C₆)alkyl or trifluoromethyl;

R⁶ and R⁷ taken together with the carbon atom to which they are attached form a ring of 4 to 8 carbon atoms; and

R⁸ is H, (C₁-C₆)alkyl, hydroxy(C₁-C₆)alkyl, benzyl (optionally substituted with benzyloxy or phenyloxy), naphthylmethyl (optionally substituted with one or more R¹), phenyl(C₂-C₆)alkyl (optionally substituted with one or more R¹), heteroarylmethyl (optionally substituted with R¹), cycloalkyl, cycloalkenyl, cycloalkylmethyl (where any carbon atom can be optionally replaced with N, S, or O and where said cycloalkylmethyl can be optionally substituted with OH, CF₃, halo, alkoxy, alkyl, benzyloxy, or alkanoyloxy), cycloalkenylmethyl (where any carbon atom can be optionally replaced with N, S, or O and where said cycloalkenylmethyl can be optionally substituted with OH, CF₃, halo, alkoxy, alkyl, benzyloxy, or alkanoyloxy);

R⁵ and R⁸, taken together with the nitrogen and carbon atoms through which they are connected form a ring of 4 to 8 carbon atoms; optionally substituted with R⁵.

In certain preferred embodiments,

R¹ is trifluoromethoxy, nitrile, alkenyl, or alkynyl;

R² is H, OH, alkyl, alkoxy, halo, or trifluoromethyl;

each R⁵ is independently (C₁-C₆)alkyl or trifluoromethyl;

R⁶ and R⁷ taken together with the carbon atom to which they are attached form a ring of 4 to 8 carbon atoms; and

R⁸ is H or (C₁-C₆)alkyl.

In certain preferred embodiments, R¹ is phenyl, naphthyl, heteroaryl, benzyloxy, phenoxy, naphthyloxy, phenylethoxy, phenoxyethoxy, phenylcarbonylamino, phenylaminocarbonyl, trifluoromethoxy, nitrile, alkenyl, alkynyl, sulfonyl, sulfonamido, alkanoyl, alkoxycarbonyl, alkylaminocarbonyl, or amino.

In certain preferred embodiments, R² is H, OH, alkyl (especially methyl, ethyl, propyl, and butyl), alkoxy (especially methoxy and ethoxy), or halo (especially chloro, fluoro, and bromo).

In certain preferred embodiments, each R⁵ in the piperazine ring is independently H or (C₁-C₆)alkyl (especially methyl, ethyl, propyl, and butyl). In certain especially preferred embodiments, each R⁵ in the piperazine ring is methyl.

In certain preferred embodiments, R⁶ and R⁷ are, independently, (C₁-C₆)alkyl (especially methyl, ethyl, propyl, and butyl) or (C₃-C₆)cycloalkyl (especially cyclopropyl, cyclobutyl, and cyclohexyl).

In certain preferred embodiments, R⁶ and R⁷ taken together with the carbon atom to which they are attached form a ring of 4 to 8 carbon atoms.

In certain preferred embodiments, R⁸ is H, (C₁-C₆)alkyl (especially methyl, ethyl, propyl, and butyl), hydroxybutyl, benzyl, naphthylmethyl, phenyl(C₂-C₆)alkyl, heteroarylmethyl, cycloalkyl (especially cyclopropyl, cyclobutyl, and cyclohexyl), cycloalkenyl, cycloalkylmethyl, cycloalkenylmethyl.

In certain preferred embodiments, R⁵ and R⁸, taken together with the nitrogen and carbon atoms through which they are connected form 4- to 8-membered heterocycloalkyl ring optionally substituted with R⁵.

Examples of R⁶ and R⁷ taken together with the carbon atom to which they are attached form a ring of 4 to 8 carbon atoms include 4, 5, or 6-membered carbon rings, e.g. a cyclohexyl ring.

Examples of R¹ include trifluoromethoxy; thienyl; phenoxy; phenylethoxy; naphthyloxy; naphthylmethoxy; naphthylethoxy; alkenyl of 2 to 6 carbon atoms; alkynyl of 2 to 6 carbon atoms; phenyl optionally substituted by one, two or three substituents selected from halo, methylenedioxy, nitrile, nitro, alkoxy of 1 to 6 carbon atoms, alkyl of 1 to 6 carbon atoms, alkenyl of 2 to 6 carbon atoms, alkynyl of 2 to 6 carbon atoms trifluoromethoxy and trifluoromethyl; and benzyloxy optionally substituted by one or two substituents selected from halo, alkoxy of 1 to 6 carbon atoms, alkyl of 1 to 6 carbon atoms and trifluoromethyl.

Examples of R² are hydrogen, halo, alkoxy of 1 to 6 carbon atoms, and hydroxy.

R⁸ may be for example H, alkyl of 1 to 6 carbon atoms, hydroxy(C₁-C₆)alkyl, benzyl, phenyl(C₂-C₆)alkyl, and cycloalkylmethyl.

Each R⁵ is, for example, selected independently from H and alkyl of 1 to 6 carbon atoms, especially methyl, ethyl, n-propyl, isopropyl, n-butyl, isobutyl, and t-butyl.

Preferred compounds of formula VII or VII* prepared by processes of the invention include:

1-{2-piperazin-1-yl-1-[3-(trifluoromethoxy)phenyl]ethyl}cyclohexanol dihydrochloride;

1-{2-(4-methylpiperazin-1-yl)-1-[3-(trifluoromethoxy)phenyl]ethyl}cyclohexanol dihydrochloride;

1-{1-[4-(benzyloxy)phenyl]-2-piperazin-1-ylethyl}cyclohexanol dihydrochloride;

1-{2-piperazin-1-yl-1-[4-(trifluoromethoxy)phenyl]ethyl}cyclohexanol dihydrochloride;

1 -{2-piperazin-1-yl-1-[3-(trifluoromethoxy)phenyl]ethyl}cyclobutanol dihydrochloride;

1-{1-[4-(benzyloxy)phenyl]-2-piperazin-1-ylethyl}cyclobutanol dihydrochloride;

1-[1-[4-(benzyloxy)phenyl]-2-(4-methylpiperazin-1-yl)ethyl]cyclobutanol dihydrochloride;

1-{2-(4-methylpiperazin-1-yl)-1-[3-(trifluoromethoxy)phenyl]ethyl}cyclobutanol dihydrochloride;

1-{1-[3-(benzyloxy)phenyl]-2-piperazin-1-ylethyl}cyclohexanol dihydrochloride;

1-[1-[3-(benzyloxy)phenyl]-2-(4-methylpiperazin-1-yl)ethyl]cyclohexanol dihydrochloride;

1-{1-[3-(benzyloxy)phenyl]-2-piperazin-1-ylethyl}cyclobutanol dihydrochloride;

1-{1-[3-(benzyloxy)phenyl]-2-piperazin-1-ylethyl}cyclobutanol dihydrochloride;

1-[1-(3′,4′-dichloro-1,1′-biphenyl-3-yl)-2-piperazin-1-ylethyl]cyclohexanol dihydrochloride;

1-[1-(3′,4′-dichloro-1,1′-biphenyl-3-yl)-2-piperazin-1-ylethyl]cyclohexanol dihydrochloride;

1-[1-(1,1′-biphenyl-3-yl)-2-piperazin-1-ylethyl]cyclohexanol dihydrochloride;

1-[1-(4′-chloro-1,1′-biphenyl-3-yl)-2-piperazin-1-ylethyl]cyclohexanol dihydrochloride;

1-[1-(4′-chloro-1,1′-biphenyl-3-yl)-2-piperazin-1-ylethyl]cyclohexanol dihydrochloride;

1-[1-(3′-chloro-1,1′-biphenyl-3-yl)-2-piperazin-1-ylethyl]cyclohexanol dihydrochloride;

1-[1-(2′-fluoro-1,1′-biphenyl-3-yl)-2-piperazin-1-ylethyl]cyclohexanol maleate;

1-[1-(3′,4′-difluoro-1,1′-biphenyl-3-yl)-2-piperazin-1-ylethyl]cyclohexanol dihydrochloride;

1-[1-(3′,4′-dichloro-1,1′-biphenyl-2-yl)-2-piperazin-1-ylethyl]cyclohexanol dihydrochloride;

1-[1-(1,1′-biphenyl-2-yl)-2-piperazin-1-ylethyl]cyclohexanol dihydrochloride;

1-[1-(3′-chloro-1,1′-biphenyl-2-yl)-2-piperazin-1-ylethyl]cyclohexanol dihydrochloride;

1-{1-[2-(1,3-benzodioxol-5-yl)phenyl]-2-piperazin-1-ylethyl}cyclohexanol dihydrochloride;

1-[1-(3′,4′-dichloro-1,1′-biphenyl-3-yl)-2-piperazin-1-ylethyl]cyclobutanol dihydrochloride;

1-[1-(3′,4′-dichloro-1,1′-biphenyl-3-yl)-2-piperazin-1-ylethyl]cyclobutanol dihydrochloride;

1-[1-(1,1′-biphenyl-4-yl)-2-(4-methylpiperazin-1-ylethyl]cyclohexanol dihydrochloride;

1-[1-(3-cyanophenyl)-2-piperazin-1-ylethyl]cyclohexanol dihydrochloride;

1-[1-(3-cyanophenyl)-2-(4-methylpiperazin-1-yl)ethyl]cyclohexanol dihydrochloride;

1-[2-piperazin-1-yl-1-(3-vinylphenyl)ethyl]cyclohexanol dihydrochloride;

1-[2-piperazin-1-yl-1-(4-vinylphenyl)ethyl]cyclohexanol dihydrochloride;

1-[2-piperazin-1-yl-1-(4-prop-1-ynylphenyl)ethyl]cyclohexanol dihydrochloride;

1-[1-(2′-chloro-1,1′-biphenyl-4-yl)-2-piperazin-1-ylethyl]cyclohexanol dihydrochloride;

1-[1-(3′-fluoro-1,1′-biphenyl-4-yl)-2-piperazin-1-ylethyl]cyclohexanol dihydrochloride;

1-[1-(3′-chloro-1,1′-biphenyl-4-yl)-2-piperazin-1-ylethyl]cyclohexanol dihydrochloride;

1-[1-(3′-cyano-1,1′-biphenyl-4-yl)-2-piperazin-1-ylethyl]cyclohexanol dihydrochloride;

1-[1-(3′-nitro-1,1′-biphenyl-4-yl)-2-piperazin-1-ylethyl]cyclohexanol dihydrochloride;

1-[1-(3′-methoxy-1,1′-biphenyl-4-yl)-2-piperazin-1-ylethyl]cyclohexanol dihydrochloride;

1-{2-piperazin-1-yl-1-[3′-(trifluoromethoxy)-1,1′-biphenyl-4-yl]ethyl}cyclohexanol dihydrochloride;

1-[1-(4′-chloro-1,1′-biphenyl-4-yl)-2-piperazin-1-ylethyl]cyclohexanol dihydrochloride;

1-[1-(3′,4′-dichloro-1,1′-biphenyl-4-yl)-2-piperazin-1-ylethyl]cyclohexanol dihydrochloride;

1-[1-(2′-chloro-1,1′-biphenyl-4-yl)-2-(4-methylpiperazin-1-yl)ethyl]cyclohexanol dihydrochloride;

1-[1-(3′-chloro-1,1′-biphenyl-4-yl)-2-(4-methylpiperazin-1-yl)ethyl]cyclohexanol dihydrochloride;

1-[1-(3′-cyano-1,1′-biphenyl-4-yl)-2-(4-methylpiperazin-1-yl)ethyl]cyclohexanol dihydrochloride;

1-[2-(4-methylpiperazin-1-yl)-1-(3′-nitro-1,1′-biphenyl-4-yl)ethyl]cyclohexanol dihydrochloride;

1-[1-(3′-methoxy-1,1′-biphenyl-4-yl)-2-(4-methylpiperazin-1-yl)ethyl]cyclohexanol dihydrochloride;

1-[1-(4′-fluoro-1,1′-biphenyl-4-yl)-2-(4-methylpiperazin-1-yl)ethyl]cyclohexanol dihydrochloride;

1-[1-(4′-methyl-1,1′-biphenyl-4-yl)-2-(4-methylpiperazin-1-yl)ethyl]cyclohexanol dihydrochloride;

1-[1-(3′-chloro-1,1′-biphenyl-4-yl)-2-piperazin-1-ylethyl]cyclobutanol dihydrochloride;

1-{2-piperazin-1-yl-1-[3′-(trifluoromethoxy)-1,1′-biphenyl-4-yl]ethyl}cyclobutanol dihydrochloride;

1-[1-(3′,4′-dichloro-1,1′-biphenyl-4-yl)-2-piperazin-1-ylethyl]cyclobutanol dihydrochloride;

1-[1-(3′,5′-dichloro-1,1′-biphenyl-4-yl)-2-piperazin-1-ylethyl]cyclobutanol dihydrochloride;

1-{(1S)-2-piperazin-1-yl-1-[3-(trifluoromethoxy)phenyl]ethyl}cyclohexanol dihydrochloride;

1-{(1R)-2-piperazin-1-yl-1-[3-(trifluoromethoxy)phenyl]ethyl}cyclohexanol dihydrochloride;

1-{(1S)-2-(4-methylpiperazin-1-yl)-1-[3-(trifluoromethoxy)phenyl]ethyl}cyclohexanol dihydrochloride;

1-{(1R)-2-(4-methylpiperazin-1-yl)-1-[3-(trifluoromethoxy)phenyl]ethyl}cyclohexanol dihydrochloride;

1-[1-(3′,4′-dichloro-1,1′-biphenyl-3-yl)-2-(4-methylpiperazin-1-yl)ethyl]cyclohexanol dihydrochloride;

1-{2-piperazin-1-yl-1-[3′-(trifluoromethoxy)-1,1′-biphenyl-3-yl]ethyl}cyclohexanol dihydrochloride;

1-{2-piperazin-1-yl-1-[4′-(trifluoromethyl)-1,1′-biphenyl-3-yl]ethyl}cyclohexanol dihydrochloride;

1-[1-(3′,4′-dimethoxy-1,1′-biphenyl-3-yl)-2-piperazin-1-ylethyl]cyclohexanol dihydrochloride;

1-{1-[6-methoxy-3′-(trifluoromethoxy)-1,1′-biphenyl-3-yl]-2-piperazin-1-ylethyl}cyclohexanol dihydrochloride;

1-[1-(3′,4′-dichloro-6-methoxy-1,1′-biphenyl-3-yl)-2-piperazin-1-ylethyl]cyclohexanol dihydrochloride;

1-{1-[6-methoxy-4′-(trifluoromethyl)-1,1′-biphenyl-3-yl]-2-piperazin-1-ylethyl}cyclohexanol dihydrochloride;

1-[1-(6-methoxy-1,1′-biphenyl-3-yl)-2-piperazin-1-ylethyl]cyclohexanol dihydrochloride;

1-[1-(3′,4′-dichloro-6-methoxy-1,1′-biphenyl-3-yl)-2-(4-methylpiperazin-1-yl)ethyl]cyclohexanol dihydrochloride;

1-[1-[6-methoxy-3′-(trifluoromethoxy)-1,1′-biphenyl-3-yl]-2-(4-methylpiperazin-1-yl)ethyl]cyclohexanol dihydrochloride;

1-[1-[6-methoxy-4′-(trifluoromethyl)-1,1′-biphenyl-3-yl]-2-(4-methylpiperazin-1-yl)ethyl]cyclohexanol dihydrochloride;

1-{1-[4-(benzyloxy)-3-(trifluoromethyl)phenyl]-2-piperazin-1-ylethyl}cyclohexanol dihydrochloride;

1-[1-[4-(benzyloxy)-3-(trifluoromethyl)phenyl]-2-(4-methylpiperazin-1-yl)ethyl]cyclohexanol dihydrochloride;

1-[1-[4-(benzyloxy)-3-bromophenyl]-2-(4-methylpiperazin-1-yl)ethyl]cyclohexanol dihydrochloride;

2-(benzyloxy)-5-[1-(1-hydroxycyclohexyl)-2-piperazin-1-ylethyl]benzonitrile dihydrochloride;

2-(benzyloxy)-5-[1-(1-hydroxycyclohexyl)-2-(4-methylpiperazin-1-yl)ethyl]benzonitrile dihydrochloride;

1-{1-[4-(benzyloxy)-3-(trifluoromethoxy)phenyl]-2-piperazin-1-ylethyl}cyclohexanol dihydrochloride;

1-{2-(4-methylpiperazin-1-yl)-1-[4-(trifluoromethoxy)phenyl]ethyl}cyclohexanol dihydrochloride;

1-{2-[4-(1,3-benzoioxol-5-ylmethyl)piperazin-1-yl]-1-[4-(trifluoromethoxy)phenyl]ethyl}cyclohexanol dihydrochloride;

1-{2-[4-(cyclohexylmethyl)piperazin-1-yl]-1-[4-(trifluoromethoxy)phenyl]ethyl}cyclohexanol dihydrochloride;

1-{2-(4-ethylpiperazin-1-yl)-1-[4-(trifluoromethoxy)phenyl]ethyl}cyclohexanol dihydrochloride;

1-{2-[cis-3,5-dimethylpiperazin-1-yl]-1-[4-(trifluoromethoxy)phenyl]ethyl}cyclohexanol dihydrochloride;

1-[1-(2′-fluoro-1,1′-biphenyl-4-yl)-2-piperazin-1-ylethyl]cyclohexanol dihydrochloride;

4′-[1-(1-hydroxycyclohexyl)-2-piperazin-1-ylethyl]-1,1′-biphenyl-2-carbonitrile dihydrochloride;

1-[1-(2′,5′-dichloro-1,1′-biphenyl-4-yl)-2-piperazin-1-ylethyl]cyclohexanol dihydrochloride;

1-{1-[4-(benzyloxy)-3-chlorophenyl]-2-piperazin-1-ylethyl}cyclohexanol dihydrochloride;

1-[1-[4-(benzyloxy)-3-chlorophenyl]-2-(4-methylpiperazin-1-yl)ethyl]cyclohexanol dihydrochloride;

1-[1-(3′-chloro-1,1′-biphenyl-4-yl)-2-(4-methylpiperazin-1-yl)ethyl]cyclobutanol dihydrochloride;

1-{2-(4-methylpiperazin-1-yl)-1-[3′-(trifluoromethoxy)-1,1′-biphenyl-4-yl]ethyl}cyclobutanol dihydrochloride;

1-[1-(3′,4′-dichloro-1,1′-biphenyl-4-yl)-2-(4-methylpiperazin-1-yl)ethyl]cyclobutanol dihydrochloride;

1-[1-(3′,5′-dichloro-1,1′-biphenyl-4-yl)-2-(4-methylpiperazin-1-yl)ethyl]cyclobutanol dihydrochloride;

1-[1-(3-ethynylphenyl)-2-piperazin-1-ylethyl]cyclohexanol dihydrochloride;

1-[1-(3-ethynylphenyl)-2-(4-methylpiperazin-1-yl)ethyl]cyclohexanol dihydrochloride;

1-[2-piperazin-1-yl-1-(3-prop-1-ynylphenyl)ethyl]cyclohexanol dihydrochloride;

1-[2-(4-methylpiperazin-1-yl)-1-(3-prop-1-ynylphenyl)ethyl]cyclohexanol dihydrochloride;

1-{2-piperazin-1-yl-1-[4-(trifluoromethoxy)phenyl]ethyl}cyclobutanol dihydrochloride;

1-[1-(4-phenoxyphenyl)-2-piperazin-1-ylethyl]cycohexanol dihydrochloride

1-[2-(4-methylpiperazin-1-yl)-1-(4-phenoxyphenyl)ethyl]cyclohexanol dihydrochloride;

1-[2-[4-(cyclohexylmethyl)piperazin-1-yl]-1-(4-phenoxyphenyl)ethyl] cyclohexanol dihydrochloride;

1-[1-(3-phenoxyphenyl)-2-piperazin-1-ylethyl]cycohexanol dihydrochloride;

1-[2-(4-methylpiperazin-1-yl)-1-(3-phenoxyphenyl)ethyl]cyclohexanol dihydrochloride;

1-[2-[4-(cyclohexylmethyl)piperazin-1-yl]-1-(3-phenoxyphenyl)ethyl] cyclohexanol dihydrochloride;

1-[2-(4-methyl-1-piperazinyl)-1-[4-phenylmethoxy)phenyl]ethyl]cyclohexanol dihydrochloride;

1-{(1R)-1-[4-(benzyloxy)-3-chlorophenyl]-2-piperazin-1-ylethyl}cyclohexanol dihydrochloride;

1-{(1S)-1-[4-(benzyloxy)-3-chlorophenyl]-2-piperazin-1-ylethyl}cyclohexanol dihydrochloride;

1-[(1R)-1-[4-(benzyloxy)-3-chlorophenyl]-2-(4-methylpiperazin-1-yl)ethyl]cyclohexanol dihydrochloride;

1-[(1S)-1-[4-(benzyloxy)-3-chlorophenyl]-2-(4-methylpiperazin-1-yl)ethyl]cyclohexanol dihydrochloride;

1-{(1S)-2-[4-(3-phenylbutyl)piperazin-1-yl]-1-[3-(trifluoromethoxy)phenyl] ethyl}cyclohexanol;

1-{(1S)-2-[(3S)-3-methylpiperazin-1-yl]-1-[3-(trifluoromethoxy)phenyl] ethyl}cyclohexanol;

1-{(1S)-2-[(3S)-3,4-dimethylpiperazin-1-yl]-1-[3-(trifluoromethoxy)phenyl] ethyl}cyclohexanol;

1-{(1S)-2-(3-ethylpiperazin-1-yl)-1-[3-(trifluoromethoxy)phenyl] ethyl}cyclohexanol;

1-{(1S)-2-[(3S)-3-ethyl-4-methylpiperazin-1-yl]-1-[3-(trifluoromethoxy) phenyl]ethyl}cyclohexanol;

1-{(1S)-2-[(3R)-3-ethyl-4-methylpiperazin-1-yl]-1-[3-(trifluoromethoxy) phenyl]ethyl}cyclohexanol;

1-[(1S)-1-(3-phenoxyphenyl)-2-piperazin-1-ylethyl]cyclohexanol;

1-[(1R)-1-(3-phenoxyphenyl)-2-piperazin-1-ylethyl]cyclohexanol;

1-{2-(4-isopropylpiperazin-1-yl)-1-[3-(trifluoromethoxy) phenyl]ethyl}cyclohexanol;

1-{(1S)-2-{4-[(1S)-1-phenylethyl]piperazin-1-yl}-1-[3-(trifluoromethoxy) phenyl]ethyl}cyclohexanol;

1-{(1S)-2-{4-[(1R)-1-phenylethyl]piperazin-1-yl}-1-[3-(trifluoromethoxy) phenyl]ethyl}cyclohexanol;

1-{(1S)-2-[(3R,5S)-3,5-dimethylpiperazin-1-yl]-1-[3-(trifluoromethoxy) phenyl]ethyl}cyclohexanol;

1-{(1S)-1-[3-(trifluoromethoxy)phenyl]-2-[(3R,5S)-3,4,5-trimethylpiperazin-1-yl]ethyl}cyclohexanol;

1-{(1S)-2-[(3R)-3-methylpiperazin-1-yl]-1-[3-(trifluoromethoxy)phenyl] ethyl}cyclohexanol;

1-{(1S)-2-[(3R)-3,4-dimethylpiperazin-1-yl]-1-[3-(trifluoromethoxy)phenyl] ethyl}cyclohexanol;

1-{(1S)-2-(octahydro-2H-pyrido[1,2-a]pyrazin-2-yl)-1-[3-(trifluoromethoxy) phenyl]ethyl}cyclohexanol;

1-{2-[4-(2-hydroxy-2-methylpropyl)piperazin-1-yl]-1-[3-(trifluoromethoxy) phenyl]ethyl}cyclohexanol;

1-{2-[4-(2-hydroxy-1,1-dimethylethyl)piperazin-1-yl]-1-[3-(trifluoromethoxy) phenyl]ethyl}cyclohexanol;

1-{1-[4-(1-naphthyloxy)phenyl]-2-piperazin-1-ylethyl}cyclohexanol;

1-{1-[4-(benzyloxy)-3-bromo-5-methoxyphenyl]-2-piperazin-1-ylethyl}cyclohexanol;

1-[1-[4-(benzyloxy)-3-bromo-5-methoxyphenyl]-2-(4-methylpiperazin-1-yl)ethyl]cyclohexanol;

1-{1-[4-(benzyloxy)-3,5-dibromophenyl]-2-piperazin-1-ylethyl} cyclohexanol;

1-[1-[4-(benzyloxy)-3,5-dibromophenyl]-2-(4-methylpiperazin-1-yl)ethyl]cyclohexanol;

(3R)-3-methyl-1-{2-piperazin-1-yl-1-[3-(trifluoromethoxy)phenyl]ethyl} cyclopentanol;

(3R)-3-methyl-1-{2-(4-methylpiperazin-1-yl)-1-[3-(trifluoromethoxy)phenyl] ethyl}cyclopentanol;

2,2-dimethyl-1-{2-piperazin-1-yl-1-[3-(trifluoromethoxy)phenyl] ethyl}cyclopentanol;

2,2-dimethyl-1-{2-(4-methylpiperazin-1-yl)-l -[3-(trifluoromethoxy)phenyl] ethyl}cyclopentanol;

1-{2-(4-methylpiperazin-1-yl)-1-[4-(1-naphthyloxy)phenyl]ethyl} cyclohexanol;

4-[1-(1-hydroxycyclohexyl)-2-piperazin-1-ylethyl]-2-(trifluoro methoxy)phenol;

4-[1-(1-hydroxycyclohexyl)-2-(4-methylpiperazin-1-yl)ethyl]-2-(trifluoromethoxy)phenol;

1-{1-[4-methoxy-3-(trifluoromethoxy)phenyl]-2-piperazin-1-ylethyl}cyclohexanol;

1-[1-[4-methoxy-3-(trifluoromethoxy)phenyl]-2-(4-methylpiperazin-1-yl)ethyl]cyclohexanol;

1-{1-[4-ethoxy-3-(trifluoromethoxy)phenyl]-2-piperazin-1-ylethyl}cyclohexanol;

1-[1-[4-ethoxy-3-(trifluoromethoxy)phenyl]-2-(4-methylpiperazin-1-yl)ethyl]cyclohexanol;

1-{1-[4-isobutoxy-3-(trifluoromethoxy)phenyl]-2-piperazin-1-ylethyl}cyclohexanol;

1-[1-[4-isobutoxy-3-(trifluoromethoxy)phenyl]-2-(4-methylpiperazin-1-yl)ethyl]cyclohexanol;

1-[1-[4-(benzyloxy)-3-(trifluoromethoxy)phenyl]-2-(4-methylpiperazin-1-yl)ethyl]cyclohexanol;

1-{1-[4-(2-phenylethyl)phenyl]-2-piperazin-1-ylethyl}cyclohexanol;

1-{2-(4-methylpiperazin-1-yl)-1-[4-(2-phenylethyl)phenyl]ethyl} cyclohexanol;

1-[(1S)-1-[4-(benzyloxy)phenyl]-2-(4-methylpiperazin-1-yl)ethyl] cyclohexanol;

1-[(1R)-1-[4-(benzyloxy)phenyl]-2-(4-methylpiperazin-1-yl)ethyl] cyclohexanol;

1-{1-[4-(benzyloxy)-3-fluorophenyl]-2-piperazin-1-ylethyl}cyclohexanol;

1-[1-[4-(benzyloxy)-3-fluorophenyl]-2-(4-methylpiperazin-1-yl)ethyl]cyclohexanol;

1-[1-(3-fluoro-4-{[4-(trifluoromethyl)benzyl]oxy}phenyl)-2-piperazin-1-ylethyl]cyclohexanol;

1-[1-(3-fluoro-4-{[4-(trifluoromethyl)benzyl]oxy}phenyl)-2-(4-methylpiperazin-1-yl)ethyl]cyclohexanol;

1-(1-{3-fluoro-4-[(4-methylbenzyl)oxy]phenyl}-2-piperazin-1-ylethyl)cyclohexanol;

1-[1-{3-fluoro-4-[(4-methylbenzyl)oxy]phenyl}-2-(4-methylpiperazin-1-yl)ethyl]cyclohexanol;

1-[1-(3-chloro-4-{[4-(trifluoromethyl)benzyl]oxy}phenyl)-2-piperazin-1-ylethyl]cyclohexanol;

1-[1-(3-chloro-4-{[4-(trifluoromethyl)benzyl]oxy}phenyl)-2-(4-methylpiperazin-1-yl)ethyl]cyclohexanol;

1-[1-(3-chloro-4-{[2-(trifluoromethyl)benzyl]oxy}phenyl)-2-piperazin-1-ylethyl]cyclohexanol;

1-[1-(3-chloro-4-{[2-(trifluoromethyl)benzyl]oxy}phenyl)-2-(4-methylpiperazin-1-yl)ethyl]cyclohexanol;

1-[1-(3-chloro-4-{[3-(trifluoromethyl)benzyl]oxy}phenyl)-2-piperazin-1-ylethyl]cyclohexanol;

1-[1-(3-chloro-4-{[3-(trifluoromethyl)benzyl]oxy}phenyl)-2-(4-methylpiperazin-1-yl)ethyl]cyclohexanol;

1-(1-{4-[(4-bromo-2-fluorobenzyl)oxy]-3-chlorophenyl}-2-piperazin-1-ylethyl)cyclohexanol;

1-[1-{4-[(4-bromo-2-fluorobenzyl)oxy]-3-chlorophenyl}-2-(4-methylpiperazin-1-yl)ethyl]cyclohexanol;

1-{1-[3-chloro-4-(2-naphthylmethoxy)phenyl]-2-piperazin-1-ylethyl} cyclohexanol;

1-{1-[4-(2-naphthylmethoxy)phenyl]-2-piperazin-1-ylethyl}cyclohexanol;

1-{2-(4-methylpiperazin-1-yl)-1-[4-(2-naphthylmethoxy)phenyl] ethyl}cyclohexanol;

1-(1-{4-[(4-bromo-2-fluorobenzyl)oxy]phenyl}-2-piperazin-1-ylethyl)cyclohexanol;

1-[1-{4-[(4-bromo-2-fluorobenzyl)oxy]phenyl}-2-(4-methylpiperazin-1-yl)ethyl]cyclohexanol;

1-[2-piperazin-1-yl-1-(4-{[4-(trifluoromethyl)benzyl]oxy}phenyl) ethyl]cyclohexanol;

1-[2-(4-methylpiperazi n-1-yl)-1-(4-{[4-(trifluoromethyl)benzyl]oxy} phenyl)ethyl]cyclohexanol;

1-[2-piperazin-1-yl-1-(4-{[3-(trifluoromethyl)benzyl]oxy}phenyl) ethyl]cyclohexanol;

1-[2-(4-methylpiperazi n-1-yl)-1-(4-{[3-(trifluoromethyl)benzyl]oxy} phenyl)ethyl]cyclohexanol;

1-[2-piperazin-1-yl-1-(4-{[2-(trifluoromethyl)benzyl]oxy} phenyl)ethyl]cyclohexanol;

1-[2-(4-methylpiperazin-1-yl)-1-(4-{[2-(trifluoromethyl)benzyl] oxy}phenyl)ethyl]cyclohexanol;

1-{1-[4-(benzyloxy)-3-methoxyphenyl]-2-piperazin-1-ylethyl}cyclohexanol;

1-[1-[4-(benzyloxy)-3-methoxyphenyl]-2-(4-methylpiperazin-1-yl)ethyl] cyclohexanol;

1-{1-[3-methoxy-4-(2-naphthylmethoxy)phenyl]-2-piperazin-1-ylethyl} cyclohexanol;

1-[1-[3-methoxy-4-(2-naphthylmethoxy)phenyl]-2-(4-methylpiperazin-1-yl)ethyl]cyclohexanol;

1-(1-{4-[(4-bromo-2-fluorobenzyl)oxy]-3-methoxyphenyl}-2-piperazin-1-ylethyl)cyclohexanol;

1-[1-4-[(4-bromo-2-fluorobenzyl)oxy]-3-methoxyphenyl-2-(4-methylpiperazin-1-yl)ethyl]cyclohexanol;

1-[1-(3-methoxy-4-{[4-(trifluoromethyl)benzyl]oxy}phenyl)-2-piperazin-1-ylethyl]cyclohexanol;

1-[1-(3-methoxy-4-{[4-(trifluoromethyl)benzyl]oxy}phenyl)-2-(4-methylpiperazin-1-yl)ethyl]cyclohexanol;

1-{1-[3-chloro-4-(2-phenylethoxy)phenyl]-2-piperazin-1-ylethyl} cyclohexanol;

1-[1-[3-chloro-4-(2-phenylethoxy)phenyl]-2-(4-methylpiperazin-1-yl)ethyl]cyclohexanol;

1-(1-{3-chloro-4-[(3-methoxybenzyl)oxy]phenyl}-2-piperazin-1-ylethyl)cyclohexanol;

1-[1-{3-chloro-4-[(3-methoxybenzyl)oxy]phenyl}-2-(4-methylpiperazin-1-yl)ethyl]cyclohexanol;

1-(1-{3-chloro-4-[(2-methoxybenzyl)oxy]phenyl}-2-piperazin-1-ylethyl)cyclohexanol;

1-[1-{3-chloro-4-[(2-methoxybenzyl)oxy]phenyl}-2-(4-methylpiperazin-1-yl)ethyl]cyclohexanol;

1-{1-[4-(2-phenylethoxy)phenyl]-2-piperazin-1-ylethyl}cyclohexanol;

1-{2-(4-methylpiperazin-1-yl)-1-[4-(2-phenylethoxy)phenyl]ethyl}cyclohexanol;

1-(1-{4-[2-(4-fluorophenyl)ethoxy]phenyl}-2-piperazin-1-ylethyl) cyclohexanol;

1-[1-{4-[2-(4-fluorophenyl)ethoxy]phenyl}-2-(4-methylpiperazin-1-yl)ethyl]cyclohexanol;

1-(1-{4-[2-(1-naphthyl)ethoxy]phenyl}-2-piperazin-1-ylethyl)cyclohexanol;

1-(2-(4-methylpiperazin-1-yl)-1-{4-[2-(1-naphthyl)ethoxy]phenyl} ethyl)cyclohexanol;

1-[1-{4-[2-(4-methoxyphenyl)ethoxy]phenyl}-2-(4-methylpiperazin-1-yl)ethyl]cyclohexanol;

1-[1-[4-(cyclohexylmethoxy)phenyl]-2-(4-methylpiperazin-1-yl)ethyl]cyclohexanol;

1-(2-(4-methylpiperazin-1-yl)-1-{4-[(1 R)-1-phenylethoxy]phenyl} ethyl)cyclohexanol;

1-(2-(4-methylpiperazin-1-yl)-1-{4-[(1 S)-1-phenylethoxy]phenyl} ethyl)cyclohexanol;

and

pharmaceutically acceptable salts thereof.

General Procedure

One method of preparing the compounds of formula I is shown in Scheme 1. The aldol condensation of the arylacetic acid (1) with cyclohexanone was carried out at −30° C. with freshly prepared LDA solution. The dianion of the acid was generated and then condensed with the ketone to provide hydroxyacid (2). Commercially available chiral amines, such as (−)benzylphenethylamine were reacted with (2) to give the corresponding salt. In the case of (−)-benzylphenethylamine, an 86:14 ratio of R to S isomers was crystallized initially. After recrystallization from acetonitrile the ratio improved to 98:2. The optically active hydroxyacid (2) recovered from the chiral amine salt was reacted with cis-2,6-dimethylpiperazine utilizing diisopropyl carbodiimide in the presence of HOBt, oxalyl chloride/DMF(cat.), or under BOP-mediated coupling conditions to give amide (3). Addition of (3) to RedAI®, LAIH₄, or AIH₂CI at room temperature converted the amide to the corresponding amine (4). The dihydrochloride salt of (S)-4 precipitated slowly out of a free base solution of (S)-4 in ethanol or methanol upon addition of ethereal HCl. The salt was recrystallized by dissolving in hot methanol, adding an equal volume of methyl tert-butyl ether, and cooling to room temperature.

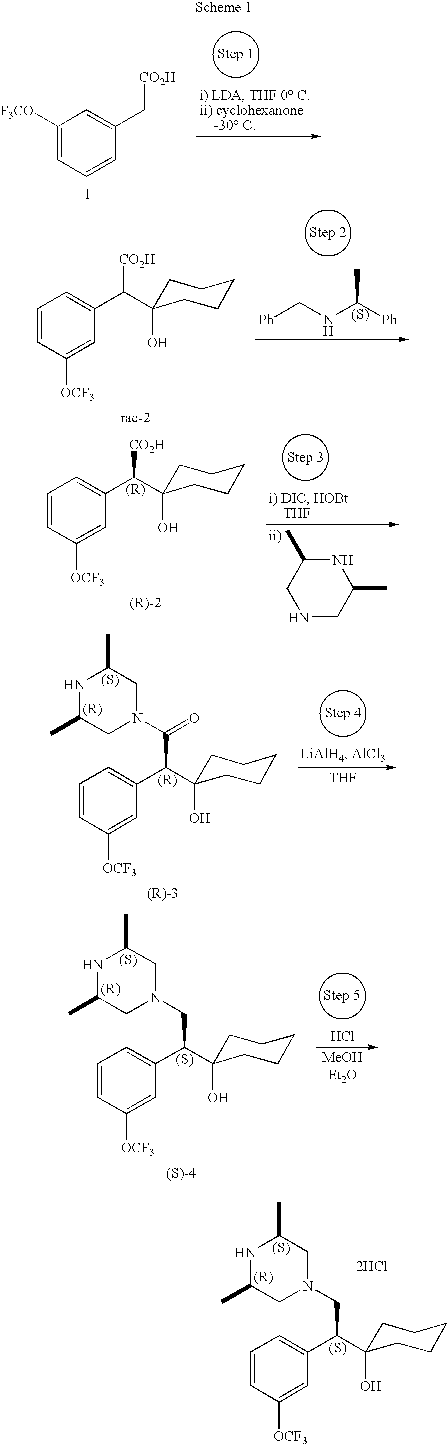

Compounds of formula I were also produced by reacting amide (6) with cyclohexanone at −30° C. with freshly prepared LDA solution to generate racemic amide (3, Scheme 2). Amide (6) in turn was prepared either by: (a) reaction of (1) with thionyl chloride and 2,6-dimethylpiperazine, or (b) by the sequence of first reacting meta-trifluoromethoxybenzaldehyde with CBr₄ and triphenylphosphine (Salaün, J. J. Org. Chem. 1977, 42, 28; Shen, W.; and Wang, L. J. Org. Chem. 1999, 64, 8873; c) Ramirez, F.; et al., J. Am. Chem. Soc. 1962, 84, 1745; and Corey, E. J. and Fuchs, P. L. Tetrahedron Lett. 1972, 3769) and then treating the formed dibromide (92% yield from meta-(trifluoromethoxy)benzaldehyde) with cis-2,6-dimethylpiperazine in a THF/H₂0 two-phase mixture in the presence of NaOH (Shen, W. and Kunzer, A. Org. Lett. 2002, 4, 1315; and Huh, D. H. et al., Tetrahedron 2002, 58, 9925). Reduction of (3) provided amine (4). Subsequent resolution with a chiral amine-resolving acid gives the desired chiral product.

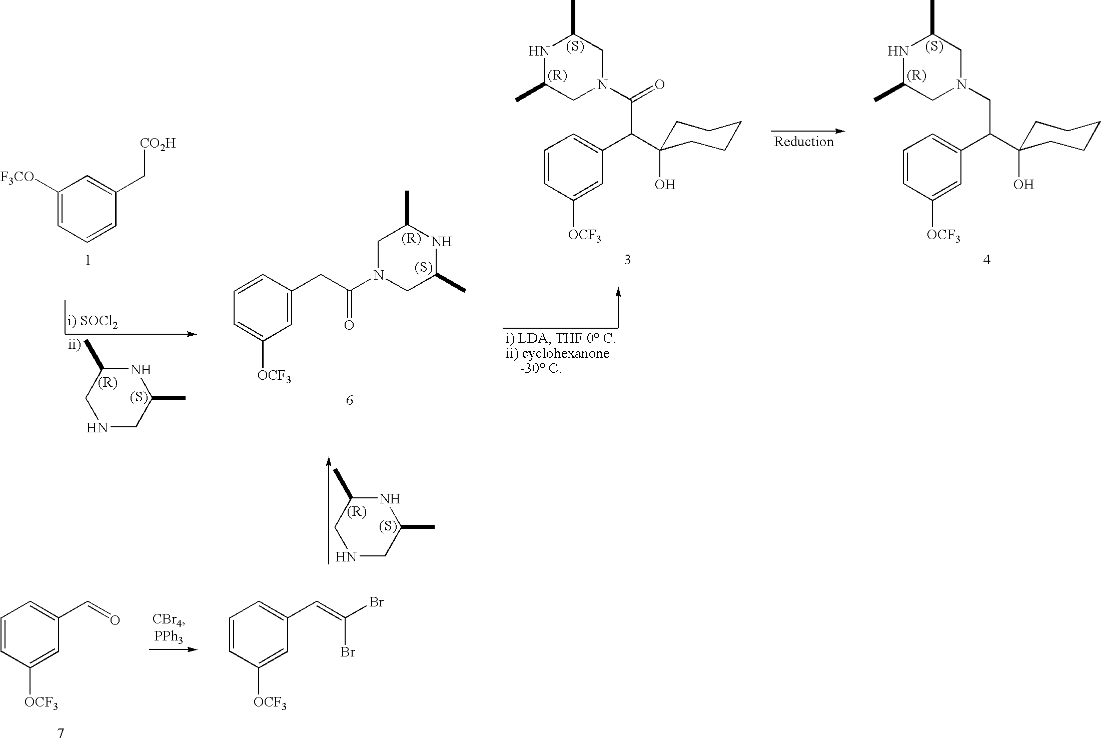

The present invention is further defined in the following Examples, in which all parts and percentages are by weight and degrees are Celsius, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### EXAMPLES

Analytical. NMR spectra of the intermediates were recorded on a Bruker Avance DPX 300 NMR spectrometer. Spectra were referenced by an internal standard. HPLC analysis of the intermediates and reaction monitoring was carried out on an Agilent 1100 liquid chromatograph equipped a Supelco 4.6×50 mm Discovery Cl 8 column. Standard method: 90:10 to 10:90 8 minute gradient of water-acetonitrile containing 0.02% TFA, flow rate 1 ml/min. LCMS data were obtained on an Agilent 1100 LC system with an Agilent 1100 LC/MS detector equipped with a 4.6×50 mm Chromolith SpeedROD column. Standard method: 90:10 to 10:90 8 minute gradient of water-acetonitrile containing 0.02%TFA, flow rate 1 ml/min. Enantiomeric purity of (1-Hydroxycyclohexyl)(3-trifluoromethoxyphenyl)acetic acid was determined by SFC on a Berger-SFC-Analytical chromatograph equipped with a 4.6×250 mm Chiralpak ADH column. Method: isocratic MeOH—CO₂ 15:85, flow rate 2 mL/min, temperature 40° C. Analytical instrumentation and methods used for the analysis of the final material are described below together with the analytical data. All starting materials are commercially available, unless otherwise noted.
(1-Hydroxycyclohexyl)(3-trifluoromethoxyphenyl)acetic acid

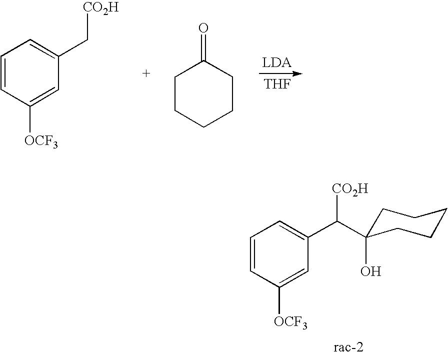

A 12-L round bottom flask equipped with a mechanical stirrer, thermocouple, nitrogen inlet, 1-L graduated addition funnel was flushed with nitrogen, the addition funnel was capped with a rubber septum. The flask was charged with tetrahydrofuran (anhydrous, Aldrich, 2.0 L) and diisopropylamine (Aldrich, 99.5%, 229.9 g, 2.26 mol). The solution in the flask was chilled to −12° C. A solution of BuLi in hexanes (2.5 M, Aldrich, 916 mL, 2.29 mol) was added slowly to the reaction mixture over a period of 2.5 hours, maintaining the temperature in the flask below −10° C. The solution in the flask was stirred at −10 to −15° C. for 30 minutes. A solution of 3-(trifluoromethoxy)phenylacetic acid (200.0 g, 0.90 mol) in 300 mL of anhydrous tetrahydrofuran was added slowly to the reaction mixture (addition time 74 min) maintaining the temperature of the reaction mixture below −10° C. The reaction mixture was stirred at −10 to −-15° C. for 45 min, then chilled to −32° C. Neat cyclohexanone (Aldrich, 133.7 g, 1.36 mol) was added slowly to the reaction mixture (addition time 38 min) maintaining the temperature range below −30° C. The reaction mixture was stirred at −30 to −40° C. for 2 hours.

A mixture of ice (200 g) was mixed with water (200 mL) and sat. aqueous solution of NH₄Cl (400 mL), and the resulting solution was added rapidly to the contents of the flask. The bi-phasic mixture was stirred rapidly for 2 minutes, then the flask was removed from the cold bath. The layers were separated and the organic layer was evaporated in vacuum. The residue was diluted with methyl tert-butly butyl ether (1.4 L). The aqueous layer was extracted with methyl tert-butyl ether (200 mL). Combined organic solutions were washed twice with 3 M aqueous HCl (600+400 mL) and then were extracted with 0.5 M aqueous NaOH solution (2×900 mL, 1×200 mL). The aqueous extracts were combined, washed with methyl tert-butyl ether (250 mL) and acidified with concentrated aqueous HCl (90 mL). The resulting white emulsion was extracted with methyl tert-butyl ether (400 mL, 2×300 mL). Combined organic extracts were washed with a mixture of water (200 mL) and brine (30 mL), then brine alone (90 mL). The resulting solution was dried with MgSO₄, suction-filtered through a paper filter and evaporated in vacuum giving a very thick yellow oil: weight 302.0 g, assayed strength 90%. Yield calculated on pure product 272 g, 95%.
(1-Hydroxycyclohexyl)(3-trifluoromethoxyphenyl)acetic acid

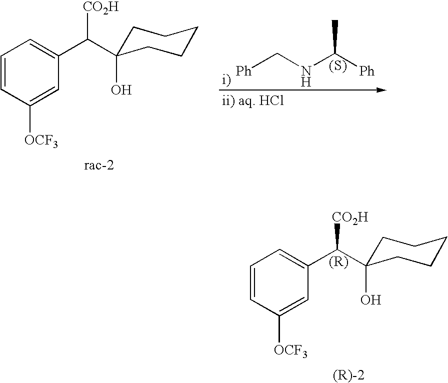

To racemic (1-hydroxycyclohexyl)(3-trifluoromethoxyphenyl)acetic acid (301.5 g, circa 90% strength, 271.3 g pure material, 0.85 mol) dissolved in acetonitrile (1.9 L) was added in one portion (S)-(−)-N-benzyl-α-methylbenzylamine (105.3 g, 0.5 mol). The clear solution was seeded with an enantiomerically pure sample (50 mg) of the salt and stirred at ambient temperature for 4 hours. The fine suspension was cooled in ice to about 2-3° C. and was left stirring in an ice bath for 15 hours. The temperature rose to about 15° C. over that period. The suspension was cooled again to 2-3° C., stirred for 2 hours, filtered, and the filter cake was washed with cold acetonitrile (2×150 mL) to give a white solid. Yield 165 g (36.5% from the amount of the racemic acid), enantiomeric purity 92% ee. The isolated solid material (165 g) was dissolved in hot (70° C.) acetonitrile (1.75 L). The clear solution was allowed to cool to ambient temperature over 15 hour period. (The crystallizing salt initially formed a thick suspension, which became substantially less viscous as the precipitate aged). The crystalline material was filtered and the filter cake was washed with cold acetonitrile (2×150 mL) to give a white solid compound (158.8 g, 35% from the racemic acid, ee 99%).

The salt (158.2 g) was dissolved in a mixture of 0.5 M hydrochloric acid (800 mL, 0.40 mol) and methyl tert-butyl ether (800 mL). The aqueous phase was separated and extracted with methyl tert-butyl ether (2×300 mL). The combined organic solutions were washed with 0.5 M hydrochloric acid (5×200 mL), brine (200 ml), dried over magnesium sulfate, filtered, and vacuum concentrated to give an oil (R)-2, which solidified upon standing to a white solid (94 g, 99% yield from the recrystallized salt).
(3R*,5S*)-3,5-Dimethyl-1-{(2R)-2-(1-hydroxycyclohexan-1-yl)-2-(3-trifluoro methoxyphenyl)acetyl} piperazine.

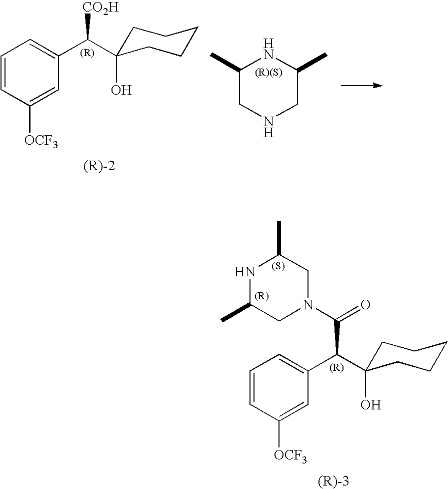

(R)-(1-Hydroxycyclohexyl)(3-trifluoromethoxyphenyl)acetic acid (93.6 g, 295.3 mmol) and HOBt hydrate (Aldrich, 61.2 g, 354.4 mmol, 1.20 equiv.) were placed into a 3-L 3-neck round bottom flask equipped with a 250-mL addition funnel, thermocouple and a mechanical stirrer. methyl tert-butyl ether (reagent grade, 1000 mL) was added and the resulting solution was cooled to 1° C. Diisopropylcarbodiimide (DIC) (Aldrich, 41.0 g, 50.0 mL, 324.9 mmol, 1.10 equiv.) was mixed with 100 mL of tetrahydrofuran and the solution was added slowly to the reaction mixture maintaining the temperature in the flask below 5° C. The ice bath was then removed and stirring was continued for 1.5 hours (temp. range: 2 to 19° C.). The reaction flask contents were cooled to 3° C. A solution of cis-dimethylpiperazine (TCI, 40.5 g, 354.4 mmol, 1.20 equiv.) in 200 mL of tetrahydrofuran and 20 mL of water was added slowly to the reaction mixture maintaining the temperature below 5° C. The bath was once again removed and the reaction mixture was stirred at room temperature for 2.5 hours (reaction was monitored by HPLC). Water (300 mL) was added to the reaction mixture and the resulting clear solution was concentrated in vacuum until organic and aqueous phases separated. The residue was diluted with a mixture of methyl tert-butyl ether (500 mL) and heptane (500 mL). 1 M aqueous. NaOH solution (400 mL) was added. The precipitate was filtered off and the solids were washed with a 1:1 mixture of methyl tert-butyl ether-heptane (300 mL). The layers of the filtrate were separated. The aqueous layer was extracted with 150 mL of 1:1 methyl tert-butyl ether—heptane mixture. The combined organic solutions were washed with 0.5 M aqueous NaOH solution (2×200 mL), brine (200 mL) and then dried with Na₂SO₄. The drying agent was filtered off and washed with a 1:1 methyl tert-butyl ether—heptane mixture (450 mL). The filtrate was evaporated in vacuum until the distillate was no longer collecting. The residue (125.3 g) was taken to the next step without further purification.
1-{(2S)-2-[(3R*,5S*)-3,5-Dimethylpiperazin-1-yl]-1-(3-trifluoromethoxyphenyl) ethyl}cyclohexanol (as a dihydrochloride)

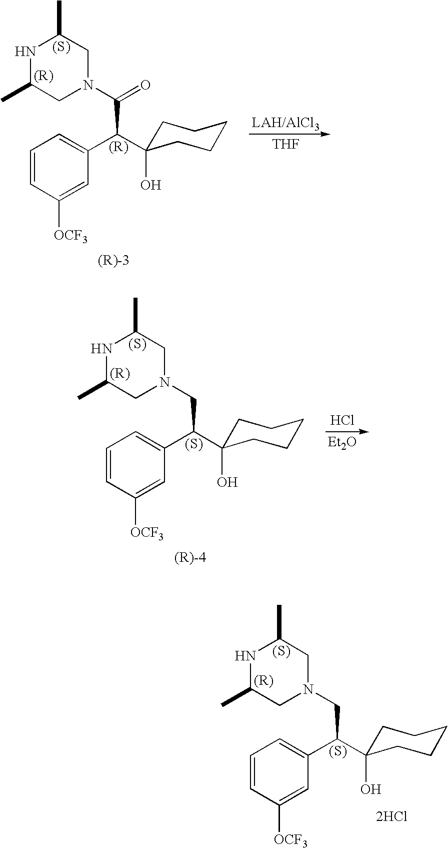

A 5-L 3-necked round bottomed flask equipped with a 1000-mL addition funnel, thermocouple and wide-blade mechanical stirrer was purged with nitrogen. tetrahydrofuran (anhydrous, 800 mL) was placed into the flask. Granular AICl₃ (Fluka, 98.3 g, 737 mmol) was added portionwise to tetrahydrofuran in the flask (Exotherm!) keeping the temperature of the reaction mixture below 40° C. The clear or slightly cloudy solution of AlCl₃ was chilled to 1° C. (some of AlCl₃—THF complex may precipitate out of solution upon cooling). LiAIH₄ solution in tetrahydrofuran (Aldrich, 1 M, 738 mL, 738 mmol) was added slowly to the reaction mixture (mild exotherm, gas evolution). The resulting clear solution was stirred at 0° C. for 40 minutes. (3R*,5S*)-3,5-Dimethyl-1-{(2R)-2-(1-hydroxycyclohexan-1-yl)-2-(3-trifluoromethoxyphenyl)acetyl}piperazine (295 mmol) was dissolved in 300 mL of anhydrous tetrahydrofuran and the solution was added slowly to the reaction mixture in the flask through the addition funnel keeping the temperature in the flask below 8° C. The stirring was then continued at room temperature for 3 hours (monitored by HPLC).

The reaction mixture was chilled to 0° C. in an ice bath, a 10 M aqueous solution of NaOH (50 mL) was added to the reaction mixture by 5-10 mL portions (Exotherm! Hydrogen evolution!). Each next portion was added only when hydrogen evolution from the previous portion slowed down and the temperature of the reaction mixture peaked and started to decrease. The temperature during the quench was maintained below 20° C. When gas evolution ceased, 520 mL of 10 M NaOH solution was added by 20-mL portions (Exotherm!). The reaction mixture thickened drastically at one point during the addition (stirrer's rpm's had to be increased) but after all of the NaOH solution was added, sticky semi-solid aluminates separated from the clear tetrahydrofuran solution.

The tetrahydrofuran solution was decanted off, and the residue was washed with methyl tert-butyl ether (2×500 mL). The tetrahydrofuran solution was evaporated in vacuum. The oily residue was mixed with the methyl tert-butyl ether extracts. The solution was washed with 1 M aqueous NaOH solution (300 mL), brine (200 mL), then it was dried with Na₂SO₄. The drying agent was filtered off and the filtrate was evaporated in vacuum. The residue was dissolved in 400 mL of methanol and the solution was evaporated in vacuum. The residue was dissolved in 400 mL of methanol. Diethyl ether (100 mL) was added to the solution. With mechanical stirring, 2 M solution of HCl in diethyl ether (Aldrich, 295 mL) was added rapidly (Exotherm!). Crystalline precipitate started to separate from the clear solution within minutes. The slurry was stirred overnight at room temperature. The precipitate was collected by filtration on a paper filter, washed with 1:1 mixture of diethyl ether-methanol (200 mL), then pure diethyl ether (100 mL) and dried on the filter in a stream of air for 1 hour: 114.9 g, (82% from the resolved acid).

The isolated solid (173.7 g, 0.37 mol) was placed into a 5-L 3-necked round-bottomed flask equipped with a temperature probe and a mechanical stirrer. The flask was placed into a heating mantle. Methanol (1.65 L) was added and the slurry was heated to 60° C. at which point all the solid dissolved and a clear solution resulted. The heat was turned off and methyl tert-butyl ether (1.65 L) was added to the solution (solution temperature went down to 44° C., the solution still remained clear). The solution was allowed to cool to room temperature. Crystallization began in 10 min (solution temp. 42° C.) and proceeded very slowly. In 30 minutes the mixture became very thick, but became much less viscous as the precipitate aged. The slurry was left stirring overnight at room temperature. Then it was filtered and washed with a 2:1 mixture of methyl tert-butyl ether-methanol (300 mL). The filter cake was dried on the filter in the stream of air for 3 hr. Then it was transferred into a crystallizing dish and further dried in vacuum oven at 55° C. for 20 hr. Final yield 157 g (91 % from the crude dihydrochloride salt) as a white fine-crystalline solid.

Analytical purity 99.8% (215 nm). Method: Prodigy ODS3 4.6×150 mm column, mobile phase acetonitrile-water (0.02% TFA), gradient 10:90 to 100:0 over 90 min, flow rate 1 mL/min.

Enantiomeric purity: >99% ee (215 nm). Method: column OD-H, flow rate 2 mL min, isocratic IPA (10%), CO₂ (90%), DEA additive. Distomer 6.1 min, eutomer 5.4 min.

¹H NMR (D₂0, 400 MHz), δ 7.52 (t, J=8.1 Hz, 1H), 7.44-7.35 (m, 3H), 3.93-3.78 (m, 3H), 3.72-3.56 (m, 3H), 3.23 (dd, J=3.0, 10.9 Hz, 1 H), 3.04 (td, J=5.3, 12.8 Hz, 2H), 1.77 (d, J=12.8 Hz, 1H), 1.59-1.04 (m, 15H), 1.36 (d, J=6.8 Hz), 1.29 (d, J=6.5 Hz).

¹³C NMR (D₂O, 100 MHz), δ 152.1, 142.2, 133.4, 123.3 (q, J=256 Hz), 123.7, 76.6, 61.5, 57.3, 55.9, 53.5, 51.8, 51.7, 38.1, 36.7, 27.7, 24.2, 23.9, 17.9, 17.8.

Found C 53.48%, H 7.41%, N 5.83%, Cl 15.19%. Theor. C 53.28%, H 7.03%, N 5.92%, Cl 14.98%. HRMS, m/z M+H: 401.24190 (calc'd for C₂₁H₃₂F₃N₂O₂ 401.24104).

The disclosures of each patent, patent application and publication cited or described in this document are hereby incorporated herein by reference, in their entireties.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed:

1. A process for preparing a substituted aryl cycloalkanol compound of formula VII*, comprising the steps of:
a) reacting a phenylacetic acid of formula I: 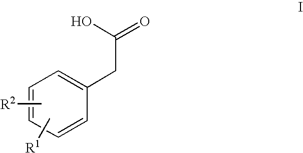
with a ketone of formula II: 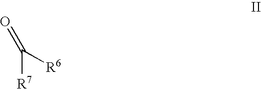
in the presence of a base to provide an acid compound of formula III: 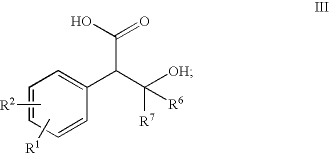
b) resolving the acid compound of formula III with an acid-resolving chiral amine to provide an acid compound of formula III*: 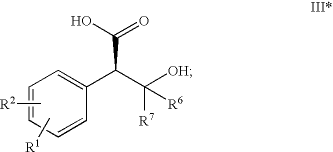
c) reacting the compound of formula III* with a piperazine compound of formula V: 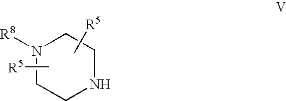
in the presence of a coupling reagent to provide an amide compound of formula VI*: 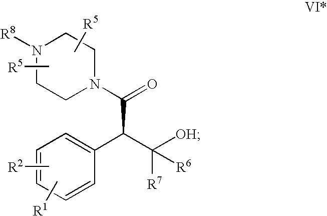
d) reacting the amide compound of formula VI* with an amide reducing agent to provide an amine compound of formula VII*: 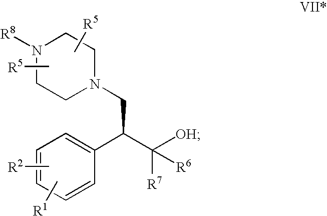
wherein:
R¹ is phenyl, naphthyl, heteroaryl, benzyloxy, phenoxy, naphthyloxy, phenylethoxy, phenoxyethoxy, naphthylmethoxy, naphthylethoxy, phenylcarbonylamino, phenylaminocarbonyl, trifluoromethoxy, nitrile, alkenyl, alkynyl, sulfonyl, sulfonamido, alkanoyl, alkoxycarbonyl, alkylaminocarbonyl, or amino;
where said phenyl, naphthyl, heteroaryl, benzyloxy, phenoxy, naphthyloxy, phenylethoxy, phenoxyethoxy, naphthylmethoxy, naphthylethoxy, phenylcarbonylamino, and phenylaminocarbonyl are optionally substituted with one or more substituents as defined for R²;
R² is H, or one or two substituents, the same or different selected from the group consisting of OH, alkyl, alkoxy, halo, trifluoromethyl, alkanoyloxy, methylenedioxy, trifluoromethoxy, nitrile, nitro, alkenyl, alkynyl, sulfonyl, and sulfonamido;
each R⁵ is independently H, (C₁-C₆)alkyl, or trifluoromethyl;
R⁶ and R⁷ are, independently, (C₁-C₆)alkyl optionally substituted with R⁵ or OH, or (C₃-C₆)cycloalkyl optionally substituted with R⁵ or OH;
or R⁶ and R⁷, taken together with the carbon atom to which they are attached, form a 4- to 8-membered cycloalkyl ring optionally substituted with R⁵ or OH,
or R⁶ and R⁷, taken together with the carbon atom to which they are attached, form a 4- to 8-membered cycloalkyl ring fused to a 4- to 6-membered cycloalkyl ring, wherein either or both of said cycloalkyl rings is optionally substituted with R⁵ or OH,
where any carbon atom of said R⁶ and R⁷ may be optionally replaced with N, S, or O;
R⁸ is H, (C₁-C₆)alkyl, hydroxy(C₁-C₆)alkyl, benzyl (optionally substituted with benzyloxy or phenyloxy), naphthylmethyl (optionally substituted with one or more R¹), phenyl(C₂-C₆)alkyl (optionally substituted with one or more R¹), heteroarylmethyl (optionally substituted with R¹), cycloalkyl, cycloalkenyl, cycloalkylmethyl (where any carbon atom can be optionally replaced with N, S, or O and where said cycloalkylmethyl can be optionally substituted with OH, CF₃, halo, alkoxy, alkyl, benzyloxy, or alkanoyloxy), cycloalkenylmethyl (where any carbon atom can be optionally replaced with N, S, or O and where said cycloalkenylmethyl can be optionally substituted with OH, CF₃, halo, alkoxy, alkyl, benzyloxy, or alkanoyloxy);
or R⁵ and R⁸, taken together with the nitrogen and carbon atoms through which they are connected, form a 4- to 8-membered heterocycloalkyl ring; said heterocycloalkyl ring optionally substituted with R⁵.

2. The process of claim 1 further comprising the step of reacting the compound of formula VII*: 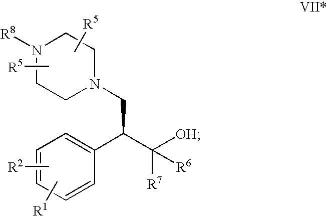
with hydrochloric acid to form a hydrochloride salt of the compound of formula VII*.

3. The process of claim 2, wherein the hydrochloride salt is the dihydrochloride salt of the compound of formula VII*.

4. The process of claim 2 further comprising the step of re-crystallizing of the hydrochloride salt of the compound of formula VII* from a solvent comprising an alcohol or alcohol-ether mixture.

5. The process of claim 4, wherein the alcohol-ether mixture comprises methyl tertiary butyl ether and methanol.

6. The process of claim 1, wherein the base in step a) is MH, MNR⁹R⁹, alkyl lithium, or aryl lithium, or any combination thereof;
wherein:
M is sodium, potassium or lithium; and
each R⁹ is independently H, alkyl, Si(alkyl)₃.

7. The process of claim 1, wherein the acid-resolving chiral amine in step b) is (S)-methylbenzylamine, (R)-methylbenzylamine, D-(+)-aminobutanol, (+)-dehydroabiethylamine, (−)-ephedrine, (−)-pseudoephedrine, norephedrine, (−)-cinchonidine, brucine, (+)-benzylphenethylamine, (−)-benzylphenethylamine, (−)-(alpha-phenylpropyl)amine, (+)-2-aminoethanol, or quinidine.

8. The process of claim 1, wherein the acid-resolving chiral amine in step b) is (S)-methylbenzylamine, (R)-methylbenzylamine, (+)-dehydroabiethylamine, (+)-benzylphenethylamine, or (−)-benzylphenethylamine.

9. The process of claim 1, wherein the acid-resolving chiral amine in step b) is (S)-methylbenzylamine, (+)-dehydroabiethylamine, or (−)-benzylphenethylamine.

10. The process of claim 1, wherein in step b) after resolving the compound of formula III the (S)-isomer of formula III* is in enantiomeric excess of at least about 20%.

11. The process of claim 1, wherein in step c) the coupling reagent is benzotriazol-1-yloxy-tris(dimethylamino)phosphonium hexa fluorophosphate (BOP), a carbodiimide, or a carbodiimide with 1-hydroxybenzotriazole.

12. The process of claim 1, wherein in step d) the amide reducing agent is borane, bis-(2-methoxyethoxy)aluminum hydride, allane, AlH₂Cl, a chloroaluminum hydride, lithium aluminum hydride, or DIBAL, or a mixture thereof.

13. The process of claim 1, wherein R¹ is trifluoromethoxy and R² is H.

14. The process of claim 13, wherein the compound of formula I is:

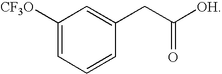

15. The process of claim 1, wherein each R⁵ in the piperazine ring is (C₁-C₆)alkyl.

16. The process of claim 1, wherein each R⁵ in the piperazine ring is methyl.

17. The process of claim 1, wherein R⁶ and R⁷, taken together with the carbon atom to which they are attached form a ring of 4 to 8 carbon atoms.

18. The process of claim 1, wherein R⁶ and R⁷, taken together with the carbon atom to which they are attached form a ring of 6 carbon atoms.

19. The process of claim 1, wherein R⁸ is H.

20. A process for preparing a substituted aryl cycloalkanol compound of formula VII* as defined in claim 1, comprising the steps of:
a) reacting a phenylacetic acid of formula I: 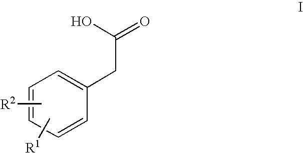
with a coupling agent and a piperazine compound of formula V: 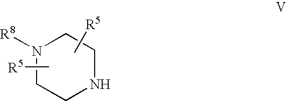
to provide an amide compound of formula VII: 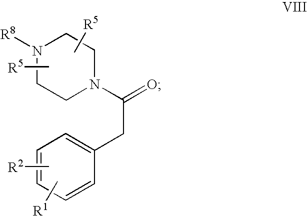
b) reacting the amide compound of formula VII with a ketone of formula II: 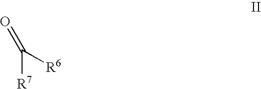
in the presence of a base to provide an amide compound of formula VI: 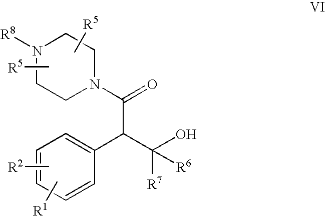
c) reacting the amide compound of formula VI with an amide reducing agent to provide an amine compound of formula VII: 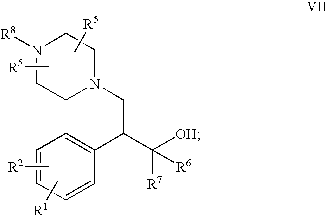
d) resolving the amine compound of formula VII with an amine-resolving chiral acid to provide an amine compound of formula VII*:

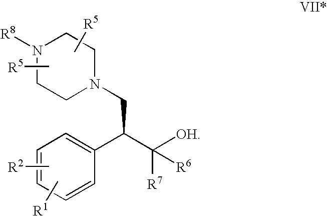

21. The process of claim 20, wherein in step a) the coupling agent is thionyl chloride, a carbodiimide, a carbodiimide with 1-hydroxybenzotriazole, (benzotriazol-1-yloxy)tris(dimethylamino)phosphonium hexafluorophosphate (BOP), (benzotriazol-1-yloxy)tripyrrolidinophosphonium hexafluorophosphate (PyBOP), or a mixture thereof.

22. A process for preparing a substituted aryl cycloalkanol compound of formula VII* as defined in claim 1, comprising the steps of:
a) reacting a benzaldehyde of formula IX: 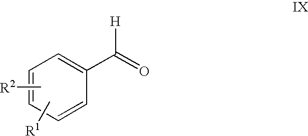
with carbon tetrabromide and a triaryl phosphine to provide a dibromoalkene compound of formula X: 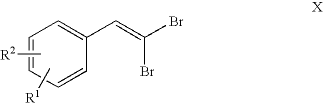
b) reacting the dibromoalkene compound of formula X with a piperazine compound of formula V: 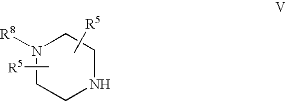
to provide an amide compound of formula VIII: 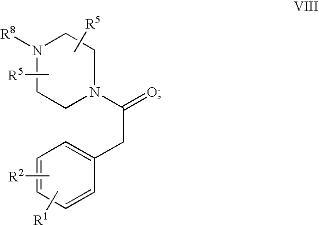
c) reacting the amide compound of formula VIII with a ketone of formula II: 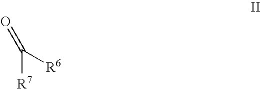
in the presence of a base to provide an amide compound of formula VI: 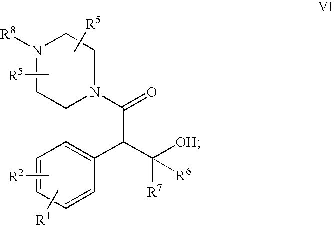
d) reacting the amide compound of formula VI with an amide reducing agent to provide an amine compound of formula VII: 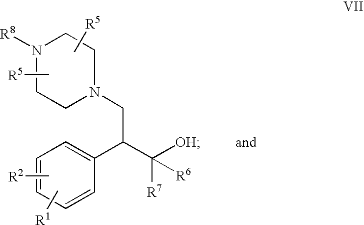
e) resolving the amine compound of formula VII with an amine-resolving chiral acid to provide an amine compound of formula VII* [chemical structure]
